(19) United States
(12) Patent Application Publication
(10) Pub. No.: US 2015/0010598 A1
(43) Pub. Date: Jan. 8, 2015

(54) COMPOSITION FOR TRANSDERMAL DELIVERY AND METHODS THEREOF

(71) Applicant: UNIVERSITY OF TENNESSEE RESEARCH FOUNDATION, Knoxville, TN (US)

(72) Inventors: Hassan Almoazen, Memphis, TN (US); Hao Lou, Memphis, TN (US); Richard Helms, Memphis, TN (US); Catherine Crill, Memphis, TN (US)

(21) Appl. No.: 14/486,903

(22) Filed: Sep. 15, 2014

Related U.S. Application Data
(63) Continuation-in-part of application No. PCT/US13/31052, filed on Mar. 13, 2013.
(60) Provisional application No. 61/610,250, filed on Mar. 13, 2012.

(51) Int. Cl. A61K 33/18; A61K 47/34; A61K 47/22; A61K 47/10; A61K 9/107; A61K 9/00

(52) U.S. Cl. CPC A61K 33/18; A61K 9/1075; A61K 9/0014; A61K 47/22; A61K 47/10; A61K 47/34
USPC 424/400; 424/670

(57) ABSTRACT

Embodiments of the presently-disclosed subject matter include compositions that include iodine or salts thereof as well as methods administering the same to subjects in need thereof. In some embodiments the compositions comprise an aqueous solution, a surfactant, a cosurfactant, an oil, and iodine or salts thereof. The composition can be an emulsion. Methods for administration include topical administration, and in some embodiments the compositions can be administered transdermally by applying the composition directly to the skin of a subject. In some embodiments the compositions are topically administered with a spray applicator that dispenses a predetermined amount of the composition upon being actuated.

### RELATED APPLICATIONS

This application is a continuation-in-part of International Patent Application No. PCT/US2013/031052, filed Mar. 13, 2013, which claims the benefit of U.S. Provisional Patent Application No. 61/610,250, filed Mar. 13, 2012, the entire disclosures of which are incorporated herein by this reference in their entirety.

### TECHNICAL FIELD

The presently-disclosed subject matter generally relates to compositions for administering agents to a subject. In particular, embodiments of the present compositions include iodine emulsions that can be administered topically to a subject in need thereof

### INTRODUCTION

Iodide accumulates in the thyroid gland regardless of the dosing route by a processes that is regulated by sodium-iodide symporter, which transports iodide from blood into thyroid epithelial cells. Iodine is vital for the normal functionality and the production of thyroidal hormones. For example, in the thyroid gland the iodide ion is oxidized to iodine and incorporated into tyrosine to produce triiodothyronine (T3) and thyroxine (T4). This process is regulated by a negative feedback loop controlled by the thyroid stimulating hormone (TSH), which is produced and secreted by the anterior pituitary gland. In this loop, TSH stimulates the production of T3 and T4, and a reduction in the levels of T4 increases the release of TSH.

Accordingly, adequate levels of iodine are needed to prevent any complications in the endocrine thyroid system. Severe iodine deficiency induces thyroid malfunction such as endemic goiter in addition to brain damage and mental retardation. Iodine deficiency can also increase the incidence of thyroid malignancy. Furthermore, during nuclear events (e.g., nuclear meltdown) radioisotopes of iodine can pose health risks. The thyroid is particularly susceptible to radioisotopes of iodine since iodine accumulates in the thyroid gland.

Despite the availability of iodine in typical diet, iodine deficiency is considered a major health problem that affects more than two billion individuals worldwide. According to the latest national nutritional survey (NHANES III 1988-1994), 15% of the U.S female adult population is considered iodine-deficient. These serious pandemic distributions have initiated the World Health Organization (WHO) to lead a public health campaigns in many countries to incorporate iodized salt as a replacement to table salt. Typically, small amounts of iodide are used as nutritional supplements to prevent iodine deficiency, and larger doses are administrated to avoid the uptake of the iodine radioisotope potentially present in the environment.

The daily recommendation dose for iodine intake, as defined by the WHO, varies by age between 90 μg/day for children and 150 μg/day for adults. Typically, this small amount could be obtained through consumption of food having iodine, iodinated table salt, potassium iodide (KI) tablets, or a KI saturate solution (SSKI). However, taking iodine orally may not be an appropriate delivery route in specific populations, such as infants, patients who underwent surgical removal of the gastrointestinal (GI) tract, or those who suffer from intestinal absorption disorders (e.g., short bowel syndrome, Whipple's disease and Celiac disease). Infants may be particularly vulnerable to iodine deficiency because of their limited thyroidal iodine store. Those unable to ingest iodide orally have few to no other viable options for routine iodide administration.

Accordingly, there remains a need for compositions, systems, and methods for administering iodine to subjects, including subjects that cannot ingest iodine orally. Thus, compositions, systems, and methods that can administer iodine quickly, easily, and efficiently to a wide variety of subjects are highly desirable and beneficial.

### BRIEF DESCRIPTION OF THE DRAWINGS

FIG. 1 is a graph that shows pseudo-ternary phase diagrams of a composition in accordance with a first embodiment composed of oil (Capryol 90®), water, surfactant (Span 20), and cosurfactant (denatured ethanol) at various surfactant/cosurfactant (S/COS) ratios (Km). The shaded area represents the domain where the compositions is monophasic.

FIG. 2 is a graph that shows the dilution line (L20) of the first embodiment. L20 is a dilution line which connects all compositions with a fixed ratio (20/80) of Oil to S/COS. Below the approximately horizontal boundary line, mixture exists as one phase, which is represented by 1 φ. Otherwise, mixture is of multiple phases, which is represented by Mφ.

FIG. 3 is a graph that shows the change of dynamic viscosity as a function of water, which is the aqueous phase of compositions along the dilution line L20 for the first embodiment. Compositions without potassium KI loading are labeled as ▪; compositions with KI loading are labeled as [symbol] .

FIG. 4 is a graph that shows the conductivity of the first embodiment along dilution line L20 versus water content. Compositions without potassium iodide (KI) loading are labeled as ▪; compositions with KI loading are labeled as [symbol] .

FIG. 5 is a graph that shows DSC curves plotted as heat flow versus temperature. From top to bottom the curves are for compositions that include: (A) 5% water, (F) 5% water with KI, (B) 10% water, (G) 10% water with KI, (C) 15% water, (H) 15% water with KI, (D) 20% water, (I) 20% water with KI, (E) 25% water, and (J) 25% water with KI.

FIG. 6 is a graph that shows permeation profiles of KI compositions. Symbols: (▪) control (KI solution); (∇) composition (5% water); (Δ) composition (10% water); (x) composition (15% water); (◯) composition (20% water); (⋄) composition (25% water). (*) Significant difference (p<0.05) for Q₂₄ compositions (15 and 20%) and control. (**) Significant difference (p<0.02) for Q₂₄ composition (25%) and control.

FIG. 7 is a graph that shows pseudo-ternary phase diagrams of a composition in accordance with another embodiment composed of oil (Capryol 90®), water, surfactant (Span 20), and cosurfactant (ethanol) at various S/COS ratios (Km).

FIG. 8 is a graph that shows the dilution line L20 of the other embodiment (20:80 of S/COS; one-phase 1 φ; multiple-phase Mφ).

FIG. 9A is a schematic a microstructure of a composition without KI in accordance with embodiments of the presently-disclosed subject matter.

FIG. 9B is a schematic that shows a microstructure of a composition with KI in accordance with embodiments of the presently-disclosed subject matter.

FIG. 10 is a graph that shows the change of dynamic viscosity as a function of water for the other embodiment. Compositions without KI loading are labeled as ▪; compositions with KI loading are labeled as [symbol] .

FIG. 11 is a graph that shows the conductivity of the other embodiment along dilution line L20 versus water content. Compositions without KI loading are labeled as ▪; compositions with KI loading are labeled as [symbol] .

FIG. 12 is a graph that shows permeation profiles of KI compositions. Symbols: (▪) control (KI solution); (D) composition F (5% water); (x) composition G (10% water); (▾) composition H (15% water); (▴) composition I (19% water); (♦) composition J (23% water).

FIG. 13 shows, from left to right, images of iodide-starch test papers that include the composition at 0 week, the composition at 2 weeks, the composition at 4 weeks, 0.025 mg/mL iodine, 0.25 mg/mL iodine, 0.5 mg/mL iodine, and 1.0 mg/mL iodine.

FIG. 14 is an image of an area on the back of the neck of a rat for administering an embodiment of the presently-disclosed compositions.

FIG. 15A is a graph showing the concentration of T3 in subjects fed healthy levels of iodine (Control Group) and in subjects fed an iodine deficient diet (ID Group) before and after treatment with an iodine composition in accordance with an embodiment of the presently-disclosed subject matter.

FIG. 15B is a graph showing the concentration of T4 in the subjects shown in FIG. 15A.

FIG. 15C is a graph showing the concentration of TSH in the subjects shown in FIG. 15A.

FIG. 16 is a graph showing the urinary iodide concentration of in the subjects shown in FIG. 15A.

FIG. 17 is a graph showing weight of the subjects shown in FIG. 15A.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

References herein to “one embodiment” of the presently-disclosed subject matter include one or more such embodiments, aspects or versions, unless the context clearly dictates otherwise.

Following long-standing patent law convention, the terms “a”, “an”, and “the” refer to “one or more” when used in this application, including the claims. Thus, for example, reference to “a surfactant” includes a plurality of such surfactants, and so forth.

Unless defined otherwise, all technical and scientific terms used herein have the same meaning as commonly understood by one of ordinary skill in the art to which the presently-disclosed subject matter pertains. Although a number of methods and materials similar or equivalent to those described herein can be used in the practice of the presently-disclosed subject matter, the preferred materials and methods are described herein.

The terms “comprising”, “including,” and “having” are intended to be inclusive and mean that there may be additional elements other than the listed elements.

Embodiments of the presently-disclosed subject matter comprise compositions that can be utilized as dermal drug delivery compositions, such as a topical, transdermal, or transmucosal compositions. In some embodiments the compositions comprise an active agent such as iodine or a salt thereof. Thus, embodiments of compositions can be utilized for administering locally or systemically therapeutically effective amounts of the active agent. Exemplary topical compositions are vicious spreadable solutions such as, but not limited to, liquids, creams, lotions, salves, pastes, balms, gels and ointments. Exemplary transdermal or transmucosal compositions can also be administered with a patch that can be adhered to a subject.

Topical administration, which can include transdermal and/or transmucosal administration, can permit controlled release of an active agent into a subject without directly invading the subject's body. This mode of administration can conveniently and effectively deliver active agent doses in a passive and continuous manner over the course of hours, days, or weeks. Typically, a transdermal active agent delivery composition can be placed anywhere on the skin, including sites typically concealed by clothing, and is therefore discreet and cosmetically elegant. Its ease of use also increases subject compliance with active agent administration. For example, a subject does not have to adhere to a strict oral regimen, perform routine injections, or travel to a clinic for treatment.

Embodiments of the active agent delivery systems of the presently-disclosed subject matter provide a needed alternative to oral delivery, which can be beneficial when oral absorption is compromised (i.e. short gut syndrome). Amongst some other active agent delivery systems, the embodied methods disclosed herein have many advantages, such as providing a dose conveniently, non-invasively, continuously, and without the interference of first-pass metabolism.

Without being bound by theory or mechanism, low molecular weight hydrophilic compounds, including highly ionized ones, can permeate through skin by appendage shunt pathway such as hair follicles and sweat glands. However, the total amount of active agent which can be diffused via this route is limited because of its small surface area compared to the total skin. In addition, ions may diffuse through lipid bilayer of the stratum corneum by the “aqueous” or the “pore” pathway model. In this model, pores are formed as a result of defects or imperfections in the interior structure of lipid bilayer which leads ions to travel through more rigid tortuous routes. Thus, ion diffusion through stratum corneum can be improved by altering the porosity of lipid bilayer. Various types of penetration enhancers such as water, alcohol, surfactant, fatty acid, ozone, etc., can effectively influence the porosity of the stratum corneum and further lower its resistance for chemicals.

Embodiments of the composition are multicomponent systems that comprise water (aqueous solution), an oil, a surfactant, a cosurfactant, and an active agent, such as iodine or a salt thereof. Embodiments of the presently-disclosed subject matter include transdermal delivery systems that offer advantages including: low cost and simple preparation, long term product stability, and main ingredients acting as solubilization and permeation enhancers. Accordingly, and without being bound by theory or mechanism, compositions of the presently-disclosed subject matter may change the internal structure of the lipid bilayer in the stratum corneum and enhance ion penetration.

A. Compositions

As used herein, the term “emulsion” refers to a composition containing an aqueous phase and an oil phase. In an emulsion one substance is referred to as the dispersed phase whereas the other is referred to as the continuous phase. For example, an emulsion having a dispersed phase of oil (i.e., oil droplets) and a continuous phase of water is called an oil-in-water emulation. As used herein, the term emulsion can refer to any oil-in-water (o/w) or water-in-oil (w/o) emulsion. The droplets (e.g., dispersions, particles, etc.) of the emulsion can include, without limitation, lipid structures that can form as a result of hydrophobic forces that drive apolar residues (e.g., long hydrocarbon chains) away from water and polar head groups toward water, when a water immiscible phase is mixed with an aqueous phase. Other typical lipid structures contemplated in the presently-disclosed subject matter include, but are not limited to, unilamellar, paucilamellar and multilamellar lipid vesicles, micelles and lamellar phases.

The term emulsion, as used herein, in inclusive of both “nanoemulsions” and “microemulsions.” Thus, reference to the present composition can refer to a composition that is an emulsion, a micoremulsion, a nanoemulsion, or a combination thereof. It is understood that, among other things, the terms nanoemulsion and micoemulsion generally refer to emulsions having droplets of an average diameter of less than about 1,000 nm.

In this regard, the compositions of the presently-disclosed subject matter may comprise droplets having an average diameter size of less than about 1,000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, or any combination thereof. In one embodiment, the droplets have an average diameter size greater than about 125 nm and less than or equal to about 600 nm. In a different embodiment, the droplets have an average diameter size greater than about 50 nm or greater than about 70 nm, and less than or equal to about 125 nm. In one embodiment, average droplet diameter is less than or equal to about 200 nm, less than or equal to about 150 nm, less than or equal to about 100 nm, or less than or equal to about 50 nm. Droplets can also have an average diameter of about 1 nm, 2 nm, 3 nm, 4 nm, 5 nm, 6 nm, 7 nm, 8 nm, 9 nm, 10 nm, 15 nm, 20 nm, 25 nm, 30 nm, 40 nm, or 45 nm. Preferably, the compositions of the presently-disclosed subject matter are oil-in-water or water-in-oil emulsions typically characterized by a droplet size of less than 250 nm.

Emulsion droplet size can be determined using any means known in the art, such as, for example, using laser light scattering.

As mentioned above, embodiments of the compositions of the presently-disclosed subject matter are multicomponent systems that may comprise water (aqueous solution), oil, surfactant, co-surfactant/organic solvent, and active agent (e.g., iodide).

The proportions of components in the composition will vary depending on the particular subject and application of the composition. In some embodiments, the composition comprises about 0.1 to about 50 wt % of aqueous solution, about 0.1 to about 50 wt % surfactant, about 0.1 to about 50 wt % cosurfactant, and about 0.1 to about 50 wt % oil. Thus, some embodiments can comprise about 0.1 wt %, about 5 wt %, about 10 wt %, about 15 wt %, about 20 wt %, about 25 wt %, about 30 wt %, about 35 wt %, about 40 wt %, about 45 wt %, or about 50 wt % of aqueous solution, surfactant, cosurfactant, or oil.

In specific embodiments, the composition comprises about 0.1 to about 30 wt % aqueous solution, about 25 to about 43 wt % surfactant, about 25 to about 43 wt % cosurfactant, and about 10 to about 24 wt % oil. In this regard, some embodiments can comprise about 0.1 wt %, 5 wt %, about 10 wt %, about 15 wt %, about 20 wt %, about 25 wt %, or about 30 wt % aqueous solution. Some specific embodiments can comprise about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, or about 43 wt % surfactant and/or cosurfactant. Furthermore, some specific embodiments may comprise about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, or about 24 wt % oil.

The amount of active agent will also vary depending on the subject and application of the composition. For example, compositions to protect against radiation may have relatively higher iodide concentrations relative to compositions serving nutritional needs. In some embodiments, the composition comprises about 0.1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of active agent. In other embodiments compositions can comprise about 75 mg/mL, about 100 mg/mL, about 150 mg/mL, about 200 mg/mL, about 250 mg/mL, about 300 mg/mL, about 350 mg/mL, about 400 mg/mL, about 450 mg/mL, about 500 mg/mL, or more active agent.

A-1. Aqueous Phase

A composition that is an emulsion typically contains about 5 to about 50 percent by volume (vol %) of aqueous phase. As used herein, percent by volume (vol %) is based on the total volume of an emulsion or small droplet size composition. In one embodiment, the aqueous phase is about 10 to about 40 vol %. In another embodiment, the aqueous phase is about 15 to about 30 vol %. The aqueous phase ranges from a pH of about 4 to about 10. In one embodiment the pH of the aqueous phase ranges from about 6 to about 8. The pH of the aqueous phase can be about 4, about 5, about 6, about 7, about 8, about 9, or about 10. The pH of the aqueous phase can be adjusted by addition of an acid or a base such as, for example, hydrochloric acid or sodium hydroxide or, for example, adding any mixture of weak acid or weak base with its salt to make a buffer.

The aqueous phase can comprise any type of aqueous phase including, but not limited to, water (e.g., H₂O, distilled water, purified water, water for injection, de-ionized water, tap water) and solutions (e.g., phosphate buffered saline (PBS) solution). The water can be deionized (hereinafter “DiH₂O”). In some embodiments the aqueous phase comprises phosphate buffered saline (PBS). The aqueous phase may further be sterile and pyrogen free.

A-2. Oil Phase

The oil in the composition can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

A preferred oil of the presently-disclosed subject matter is Capryol 90 ® (HLB 6; propylene glycol monocaprylate (type II) NF) (Gattefosse).

In embodiments of the presently-disclosed subject matter, the oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the co-surfactant/organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, framesol, ylangene, bisabolol, farnesene, ascaridole, chenopodium oil, citronellal, citral, citronellol, chamazulene, yarrow, guaiazulene, chamomile, semi-synthetic derivatives, or combinations thereof. In one aspect of the presently-disclosed subject matter, the volatile oil in the silicone component is different than the oil in the oil phase.

A-3. Surfactants

The surfactants of the presently-disclosed subject matter can be a pharmaceutically acceptable ionic surfactant, a pharmaceutically acceptable nonionic surfactant, a pharmaceutically acceptable cationic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Further, the surfactant can be a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant. Examples of polymeric surfactants include, but are not limited to, a graft copolymer of a poly(methyl methacrylate) backbone with multiple (at least one) polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, or combinations thereof.

Surface active agents or surfactants, are amphipathic molecules that contain a non-polar hydrophobic portion, usually a straight or branched hydrocarbon or fluorocarbon chain containing 8-18 carbon atoms, attached to a polar or ionic hydrophilic portion. The hydrophilic portion can be nonionic, ionic or zwitterionic. The hydrocarbon chain interacts weakly with the water molecules in an aqueous environment, whereas the polar or ionic head group interacts strongly with water molecules via dipole or ion-dipole interactions. Based on the nature of the hydrophilic group, surfactants are classified into anionic, cationic, zwitterionic, nonionic and polymeric surfactants.

Suitable surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Additional suitable surfactants include, but are not limited to, non-ionic lipids, such as glyceryl laurate, glyceryl myristate, glyceryl dilaurate, glyceryl dimyristate, semi-synthetic derivatives thereof, and mixtures thereof.

In additional embodiments, the surfactant is a polyoxyethylene fatty ether having a polyoxyethylene head group ranging from about 2 to about 100 groups, or an alkoxylated alcohol having the structure R₅—(OCH₂CH₂)_{y}—OH, wherein R₅ is a branched or unbranched alkyl group having from about 6 to about 22 carbon atoms and y is between about 4 and about 100, and preferably, between about 10 and about 100. Preferably, the alkoxylated alcohol is the species wherein R₅ is a lauryl group and y has an average value of 23.

In a different embodiment, the surfactant is an alkoxylated alcohol which is an ethoxylated derivative of lanolin alcohol. Preferably, the ethoxylated derivative of lanolin alcohol is laneth-10, which is the polyethylene glycol ether of lanolin alcohol with an average ethoxylation value of 10.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis(imidazoyl carbonyl)), nonoxynol-9, Bis(polyethylene glycol bis(imidazoyl carbonyl)), BRIJ 35, BRIJ 56, BRIJ 72, BRIJ 76, BRIJ 92V, BRIJ 97, BRIJ 58P, CREMOPHOR EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O—(N-heptylcarbamoyl)-alpha-D-glucopyranoside, Nonaethylene glycol monododecyl ether, N-Nonanoyl-N-methylglucamine, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN 20, SPAN 40, SPAN 60, SPAN 65, SPAN 80, SPAN 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, TRITON CF-21, TRITON CF-32, TRITON DF-12, TRITON DF-16, TRITON GR-5M, TRITON QS-15, TRITON QS-44, TRITON X-100, TRITON X-102, TRITON X-15, TRITON X-151, TRITON X-200, TRITON X-207, TRITON X-114, TRITON X-165, TRITON X-305, TRITON X-405, TRITON X-45, TRITON X-705-70, TWEEN 20, TWEEN 21, TWEEN 40, TWEEN 60, TWEEN 61, TWEEN 65, TWEEN 80, TWEEN 81, TWEEN 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products. Poloxamers are used in the composition of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable cationic surfactants include, but are not limited to, a quaternary ammonium compound, an alkyl trimethyl ammonium chloride compound, a dialkyl dimethyl ammonium chloride compound, a cationic halogen-containing compound, such as cetylpyridinium chloride, Benzalkonium chloride, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, Dimethyldioctadecylammonium bromide, Dodecylethyldimethylammonium bromide, Dodecyltrimethylammonium bromide, Dodecyltrimethylammonium bromide, Ethylhexadecyldimethylammonium bromide, Girard's reagent T, Hexadecyltrimethylammonium bromide, Hexadecyltrimethylammonium bromide, N,N′,N′-Polyoxyethylene(10)-N-tallow-1,3-diaminopropane, Thonzonium bromide, Trimethyl(tetradecyl)ammonium bromide, 1,3,5-Triazine-1,3,5(2H, 4H, 6H)-triethanol, 1-Decanaminium, N-decyl-N,N-dimethyl-, chloride, Didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, Alkyl 1 or 3 benzyl-1-(2-hydroxethyl)-2-imidazolinium chloride, Alkyl bis(2-hydroxyethyl)benzyl ammonium chloride, Alkyl demethyl benzyl ammonium chloride, Alkyl dimethyl 3,4-dichlorobenzyl ammonium chloride (100% C12), Alkyl dimethyl 3,4-dichlorobenzyl ammonium chloride (50% C14, 40% C12, 10% C16), Alkyl dimethyl 3,4-dichlorobenzyl ammonium chloride (55% C14, 23% C12, 20% C16), Alkyl dimethyl benzyl ammonium chloride, Alkyl dimethyl benzyl ammonium chloride (100% C14), Alkyl dimethyl benzyl ammonium chloride (100% C16), Alkyl dimethyl benzyl ammonium chloride (41% C14, 28% C12), Alkyl dimethyl benzyl ammonium chloride (47% C12, 18% C14), Alkyl dimethyl benzyl ammonium chloride (55% C16, 20% C14), Alkyl dimethyl benzyl ammonium chloride (58% C14, 28% C16), Alkyl dimethyl benzyl ammonium chloride (60% C14, 25% C12), Alkyl dimethyl benzyl ammonium chloride (61% C11, 23% C14), Alkyl dimethyl benzyl ammonium chloride (61% C12, 23% C14), Alkyl dimethyl benzyl ammonium chloride (65% C12, 25% C14), Alkyl dimethyl benzyl ammonium chloride (67% C12, 24% C14), Alkyl dimethyl benzyl ammonium chloride (67% C12, 25% C14), Alkyl dimethyl benzyl ammonium chloride (90% C14, 5% C12), Alkyl dimethyl benzyl ammonium chloride (93% C14, 4% C12), Alkyl dimethyl benzyl ammonium chloride (95% C16, 5% C18), Alkyl dimethyl benzyl ammonium chloride, Alkyl didecyl dimethyl ammonium chloride, Alkyl dimethyl benzyl ammonium chloride, Alkyl dimethyl benzyl ammonium chloride (C12-16), Alkyl dimethyl benzyl ammonium chloride (C12-18), Alkyl dimethyl benzyl ammonium chloride, dialkyl dimethyl benzyl ammonium chloride, Alkyl dimethyl dimethybenzyl ammonium chloride, Alkyl dimethyl ethyl ammonium bromide (90% C14, 5% C16, 5% C12), Alkyl dimethyl ethyl ammonium bromide (mixed alkyl and alkenyl groups as in the fatty acids of soybean oil), Alkyl dimethyl ethylbenzyl ammonium chloride, Alkyl dimethyl ethylbenzyl ammonium chloride (60% C14), Alkyl dimethyl isopropylbenzyl ammonium chloride (50% C12, 30% C14, 17% C16, 3% C18), Alkyl trimethyl ammonium chloride (58% C18, 40% C16, 1% C14, 1% C12), Alkyl trimethyl ammonium chloride (90% C18, 10% C16), Alkyldimethyl(ethylbenzyl) ammonium chloride (C12-18), Di-(C8-10)-alkyl dimethyl ammonium chlorides, Dialkyl dimethyl ammonium chloride, Dialkyl methyl benzyl ammonium chloride, Didecyl dimethyl ammonium chloride, Diisodecyl dimethyl ammonium chloride, Dioctyl dimethyl ammonium chloride, Dodecyl bis(2-hydroxyethyl)octyl hydrogen ammonium chloride, Dodecyl dimethyl benzyl ammonium chloride, Dodecylcarbamoyl methyl dimethyl benzyl ammonium chloride, Heptadecyl hydroxyethylimidazolinium chloride, Hexahydro-1,3,5-tris(2-hydroxyethyl)-s-triazine, Hexahydro-1,3,5-tris(2-hydroxyethyl)-s-triazine, Myristalkonium chloride (and) Quat RNIUM 14, N,N-Dimethyl-2-hydroxypropylammonium chloride polymer, n-Tetradecyl dimethyl benzyl ammonium chloride monohydrate, Octyl decyl dimethyl ammonium chloride, Octyl dodecyl dimethyl ammonium chloride, Octyphenoxyethoxyethyl dimethyl benzyl ammonium chloride, Oxydiethylenebis(alkyl dimethyl ammonium chloride), Quaternary ammonium compounds, dicoco alkyldimethyl, chloride, Trimethoxysily propyl dimethyl octadecyl ammonium chloride, Trimethoxysilyl quats, Trimethyl dodecylbenzyl ammonium chloride, semi-synthetic derivatives thereof, and combinations thereof.

Exemplary cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, or tetradecyltrimethylammonium halides. In some particular embodiments, suitable cationic halogen containing compounds comprise, but are not limited to, cetylpyridinium chloride (CPC), cetyltrimethylammonium chloride, cetylbenzyldimethylammonium chloride, cetylpyridinium bromide (CPB), cetyltrimethylammonium bromide (CTAB), cetyidimethylethylammonium bromide, cetyltributylphosphonium bromide, dodecyltrimethylammonium bromide, and tetrad ecyltrimethylammonium bromide. In particularly preferred embodiments, the cationic halogen containing compound is CPC, although the compositions of the presently-disclosed subject matter are not limited to compositions with an particular cationic containing compound. A variety of cationic surfactants are contemplated including, but not limited to dioloeyl-3-trimethylammonium propane (DOTAP) and dioleoyl-sn-glycerol-3-ethylphosphocholine (DEPC).

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecyl amine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4,1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, TRIZMA dodecyl sulfate, TWEEN 80, Ursodeoxycholic acid, semi-synthetic derivatives thereof, and combinations thereof.

Suitable zwitterionic surfactants include, but are not limited to, an N-alkyl betaine, lauryl amido propyl dimethyl betaine, an alkyl dimethyl glycinate, an N-alkyl amino propionate, CHAPS, minimum 98% (TLC), CHAPS, SigmaUltra, minimum 98% (TLC), CHAPS, for electrophoresis, minimum 98% (TLC), CHAPSO, minimum 98%, CHAPSO, SigmaUltra, CHAPSO, for electrophoresis, 3-(Decyldimethylammonio)propanesulfonate inner salt, 3-Dodecyldimethylammonio)propanesulfonate inner salt, SigmaUltra, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In some embodiments, the compositions of the presently-disclosed subject matter may comprises a cationic surfactant, which can be cetylpyridinium chloride. In other embodiments of the presently-disclosed subject matter, the composition comprises a cationic surfactant, and the concentration of the cationic surfactant is less than about 5.0% and greater than about 0.001%. In yet another embodiment of the presently-disclosed subject matter, the composition comprises a cationic surfactant, and the concentration of the cationic surfactant is selected from the group consisting of less than about 5%, less than about 4.5%, less than about 4.0%, less than about 3.5%, less than about 3.0%, less than about 2.5%, less than about 2.0%, less than about 1.5%, less than about 1.0%, less than about 0.90%, less than about 0.80%, less than about 0.70%, less than about 0.60%, less than about 0.50%, less than about 0.40%, less than about 0.30%, less than about 0.20%, or less than about 0.10%. Further, the concentration of the cationic agent in the composition is greater than about 0.002%, greater than about 0.003%, greater than about 0.004%, greater than about 0.005%, greater than about 0.006%, greater than about 0.007%, greater than about 0.008%, greater than about 0.009%, greater than about 0.010%, or greater than about 0.001%. In one embodiment, the concentration of the cationic agent in the composition is less than about 5.0% and greater than about 0.001%.

In another embodiment of the presently-disclosed subject matter, the composition comprises at least one cationic surfactant and at least one non-cationic surfactant. The non-cationic surfactant is a nonionic surfactant, such as a polysorbate (Tween), such as polysorbate 80, polysorbate 60 or polysorbate 20. In one embodiment, the non-ionic surfactant is present in a concentration of about 0.01% to about 5.0%, or the non-ionic surfactant is present in a concentration of about 0.1% to about 3%. In yet another embodiment of the presently-disclosed subject matter, the composition comprises a cationic surfactant present in a concentration of about 0.01% to about 2%, in combination with a nonionic surfactant.

A preferred surfactant of the presently-disclosed subject matter is Span 20.

A-4. Co-Surfactants/Organic Solvents

Organic solvents/co-surfactants in the compositions of the presently-disclosed subject matter include, but are not limited to, C₁₋₁₂ alcohol, diol, triol, dialkyl phosphate, tri-alkyl phosphate, such as tri-n-butyl phosphate, semi-synthetic derivatives thereof, and combinations thereof. In one aspect of the presently-disclosed subject matter, the organic solvent is an alcohol chosen from a nonpolar solvent, a polar solvent, a protic solvent, or an aprotic solvent.

Thus, examples of suitable co-surfactants for the presently-disclosed subject matter include, but are not limited to, ethanol, methanol, isopropyl alcohol, glycerol, medium chain triglycerides, acetone, dimethyl sulfoxide (DMSO), acetic acid, n-butanol, butylene glycol, perfumers alcohols, n-propanol, propylene glycols, triacetin, dichloromethane, semi-synthetic derivatives thereof, and any combination thereof.

A-5. Additional Ingredients

Additional compounds suitable for use in the compositions of the presently-disclosed subject matter include but are not limited to one or more solvents, such as an organic phosphate-based solvent, bulking agents, coloring agents, pharmaceutically acceptable excipients, a preservative, pH adjuster, buffer, chelating agent, antioxidants, etc. The additional compounds can be admixed into a previously emulsified composition, or the additional compounds can be added to the original mixture to be emulsified. In certain of these embodiments, one or more additional compounds are admixed into an existing composition immediately prior to its use.

Suitable preservatives and/or antioxidants in the compositions of the presently-disclosed subject matter include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives and/or antioxidants include, but are not limited to, benzyl alcohol, chlorhexidine (bis(p-chlorophenyldiguanido)hexane), chlorphenesin (3-(-4-chloropheoxy)-propane-1,2-diol), Kathon C G (methyl and methylchloroisothiazolinone), parabens (methyl, ethyl, propyl, butyl hydrobenzoates), phenoxyethanol(2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The composition of the presently-disclosed subject matter may further comprise at least one pH adjuster. Suitable pH adjusters in the composition of the presently-disclosed subject matter include, but are not limited to, diethyanolamine, lactic acid, monoethanolamine, triethylanolamine, sodium hydroxide, sodium phosphate, semi-synthetic derivatives thereof, and combinations thereof.

In addition, the composition of the presently-disclosed subject matter can comprise a chelating agent. In one embodiment of the presently-disclosed subject matter, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The composition of the presently-disclosed subject matter can additionally comprise a buffering agent, such as a pharmaceutically acceptable buffering agent.

Additionally, excipients may be used in the compositions of the presently-disclosed subject matter. Additionally, certain excipients can function as oil, surfactants or cosurfactant examples of the presently-disclosed subject matter. Examples include Labrafac, Transcutol P, Canola oil, Labrafac CC, Labrasol, Plurol Oleique CC 497, Miglyol 812, Labrafil, Glyceryl Dioleate, PEG 300, PEG 400, Tefose 1500, Stearyl Alcohol, Plurol Diisostearique, Labrafil M1944CS, ST-Cyclomethicone 5, Caprysol 90, Apifil, MOD, Labrafil M2130CS, Cetyl Alcohol, Gelot 64, Sedefos 75, Tefose 63, Cetostearyl alcohol, Compritol 888, Cremophor RH40, Luvigel EM, Cremophor A6, Luvigel EM, Lauroglycol FCC, Luvigel EM, Lauroglycol FCC, Labrafac PG, Luvitol EHO, Cremophor A25, Geleol, DPPG, Sefsol 218, corn oil, cottonseed oil, sesame oil, Cremophor A6, Cremophor ELP, Cremophor EL, Cremophor RH40, Cavasol W7 HP (beta-CD), Solutol HS 15, Soluphor P, Miglyol 810, Miglyol 812, Lutrol F68, Lutrol F127, Gelucire 44/14, Gelucire 50/13, vitamin E-TPGS

A-6. Active Agents

Embodiments of the presently-disclosed subject matter include a transdermal iodide delivery system which has potential therapeutic uses when oral administration is not suitable. As indicated above, iodide is vital for the biosynthesis of thyroid hormones triiodothyronine (T3) and thyroxine (T4). It has been illustrated by previous studies that thyroid gland is the location where iodide is massively accumulated regardless of the dosing route. The accumulation of iodide in the thyroid is regulated by sodium-iodide symporter which transports iodide from blood into thyroid epithelial cells. Typically, small amount of iodide (and/or any acceptable salt form thereof) is used as a nutritional supplement to prevent iodine deficiency, whereas larger doses are administrated to avoid the uptake of radioactive iodide to thyroids during nuclear fission accidents. Usually, iodide is administered orally, by using table salt enriched with iodide, KI tablet, and KI saturate solution (SSKI). However, oral administration may not be suitable for all age groups, especially for the elderly and children.

Transdermal drug delivery system is an alternative to oral delivery, especially when oral absorption is compromised (i.e. short gut syndrome). Amongst some other delivery systems, transdermal administration has many advantages such as providing a dose conveniently, non-invasively, continuously, and without the interference of first-pass metabolism.

Thus, as an example of the presently-disclosed subject matter, a w/o composition system incorporating a salt of iodine (iodide) and/or any acceptable salt form thereof. For example some embodiments comprise potassium iodide salt (KI). Several physicochemical characterizations were conducted to evaluate the system. Franz diffusion cells ware utilized to evaluate the penetration of the iodide ions through skin. In addition, fluorescence quenching method was used to pinpoint the mechanism of iodide diffusion through stratum corneum. Other examples of iodide-containing salts include, but are not limited to, sodium iodide, zinc iodide, and copper iodide.

Another embodiment of the presently-disclosed subject matter is the delivery of at least one trace/multitrace elements and salt forms thereof (and hydrates, anhydrous weak acids, weak bases, etc.) by emulsion technology. Multitrace elements are needed metal elements for meonates, infants, children, and adult development. Examples of these elements include zinc, copper, manganese, chromium, selenium, magnesium, aluminum, molybdenum, iron (ferric and ferrous), etc. Reference to any multitrace elements and/or active agents herein refers to the substance as well as salts thereof, including pharmaceutically acceptable salts thereof.

Zinc is an essential nutrient which is needed for many enzymes including carbonic anhydrase, alkaline, phosphatase, lactic dehydrogenase, and both RNA and DNA polymerase. In addition, zinc facilitates wound healing, helps maintain normal skin hydration, and senses of taste and smell. Zinc prevents developments of Parakeratosis, hypogeusia, anorexia, dysomia, geophagia, hypogonadism, growth retardation, and hepatosplenomegaly.

Copper is an important nutrient for serum ceruloplasmin, and oxidase necessary for proper formation of the iron carrier protein, transferin. Copper prevents the development of leucopenia, neutropenia, anemia, depressed ceruloplasmin levels, impaired transferin formation and secondary iron deficiency.

Manganese serves as an activator for enzymes such as polysaccharide polymerase, liver arginase, cholinesterase, and pyruvate carboxylase. Manganese prevents development of nausea, vomiting, weight loss, dermatitis, and changes in growth and color of hair.

Chromium is part of glucose tolerance factor, and activator of insulin-mediated reactions. Chromium maintains normal glucose metabolism and peripheral nerve function. Chromium prevents developments of impaired glucose tolerance, ataxia, peripheral neuropathy, and a confusional state similar to mild/moderate hepatic encephalopathy.

Selenium is utilized to support metabolism.

Additionally, in other embodiments, the nanoemulsions of the presently-disclosed subject matter can be used to deliver iron and magnesium. Iron is required fort the production of hemoglobin and myoglobin (the form of hemoglobin found in muscle tissue) requires this nutrient. Iron is also needed for the oxygenation of red blood cells, a healthy immune system and for energy production. Magnesium is a mineral that is required for the enzyme reactions that metabolize fats and carbohydrates to produce energy. Additionally, plays a role in metabolism and transport of nutrients to the cells. Magnesium is also important in the function of muscles.

As used herein, any active agent can also refer to a salt thereof. For example, the term “iodine” is inclusive of salts thereof (i.e., iodide). Thus, the terms “iodine” and “iodine or salts thereof” can be used interchangeably herein to refer to I₂, iodide, combinations thereof, and the like.

B. Delivery Mechanisms

There are several methods for administering the compositions of the presently-disclosed subject matter to and through the skin of a subject in need thereof. In some embodiments an effective amount of an active agent (e.g., iodide) can be administered from a composition transdermally. Subjects may be in need of an active agent such for a multitude of reasons. Subjects can be in need of iodide because they are deficient iodide, their diet lacks sufficient iodide, they want to be protected against radiation for iodine isotopes, and the like. In this respect, the present methods can include methods of treating a subject in need thereof.

The terms “treatment” or “treating” refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. As will be understood by those of ordinary skill upon reviewing this paper, the terms treatment and the like in some instances refer to providing a dietary supplement, such as iodide, to a subject.

An “effective amount” of an active agent (e.g., iodide) or pharmaceutical composition to be used in accordance with the presently-disclosed subject matter is intended to mean a nontoxic but sufficient amount of the agent, such that the desired prophylactic or therapeutic effect is produced. Thus, the exact amount of the active agent or pharmaceutical composition that is required will vary from subject to subject, depending on the species, age, and general condition of the subject, the severity of the condition being treated, the particular carrier or adjuvant being used and its mode of administration, and the like. Similarly, the dosing regimen should also be adjusted to suit the individual to whom the composition is administered and will once again vary with age, weight, metabolism, etc. of the individual. Accordingly, the “effective amount” of any particular active agent, or pharmaceutical composition thereof, will vary based on the particular circumstances, and an appropriate effective amount may be determined in each case of application by one of ordinary skill in the art using only routine experimentation.

Furthermore, the term “subject” is inclusive of both human and animal subjects. Thus, veterinary uses are provided in accordance with the embodiments of the presently-disclosed subject matter and certain embodiments provide methods for administering active agents in mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

B-1. Transdermal Patch

Transdermal patches are certainly known in the art. Many of these patches are suitable for use in connection with the presently-disclosed subject matter.

For example, in one embodiment of the presently-disclosed subject matter, the transdermal patch is a simple adhesive patch, which comprises an impermeable backing layer, a release liner, and a drug/adhesive containing matrix.

Typically, the impermeable backing layer defines the top of the drug delivery device, i.e., the side furthest away from the skin when the device is in use. The backing forms an occlusive layer that prevents the loss of drug and/or enhancers to the environment and protects the patch from contamination from the environment. The backing layer may be opaque so as to protect the drug from light.

The backing layer can be made from standard commercially available films for medical use, such as those supplied by 3M Corporation, St. Paul, Minn.; Dow Chemical, Midland, Mich.; or AF Packaging, Winston-Salem, N.C. Suitable materials which can be used to form the backing layer include films or sheets of polyolefin, polyester, polyurethane, polyvinyl alcohol, polyvinylidene, polyamide, ethylene-vinylacetate copolymer, ethylene-ethylacrylate copolymer, and the like, metal-vapor deposited films or sheets thereof, rubber sheets or films, expanded synthetic resin sheets or films, unwoven fabrics, fabrics, knitted fabrics, paper, and foils. These materials can be used individually or as laminates. These films can be pigmented or metalized.

Specifically, the backing layers can include Scotchpak® 1006 and 1009, skin-colored aluminized polyester films of approximately 70-80 μm in thickness, and 3M-1012, a transparent polyester film laminate, all of which are available from 3M Corporation.

In some aspects of the presently-disclosed subject matter, the patch may include a peel strip or release liner to cover the surface of the pressure-sensitive adhesive during storage, and prevent evaporative loss of the drug or enhancer(s). The release liner may be formed with dimples for decreasing contacting surface with the adhesive layer, and it may also be formed with a pull-tab for making it easier for removing it from the device.

The peel strip may be made from any impermeable film, such as is specified for the backing layer. Additionally it may be made from metal foil, Mylar™ polyethylene terephthalate, or any material normally used for this purpose in the art that is compatible with the drug and the chosen adhesive. Examples of suitable compositions for the release liner include siliconized polyester, poly (1,1-dihydroperfluoroctylmethacrylate-), fumed silica in silicone rubber, end-capped siliconized polyethylene terephthalate, polytetrafluoroethylene, cellophane, a film of polyvinyl chloride having titanium dioxide dispersed therein, and the like. Preferred release liners include a silicon coated Release Technology 381B and fluoropolymer coated polyester films, such as 3M Scotchpak® 1022 film.

B-2. Topical Compositions

The compositions of the presently-disclosed subject matter can be administered topically. The term “topical administration” as used herein refers to the delivery of a composition onto the surface of a skin or mucosal tissue of a subject, and topical administration is inclusive of transdermal administration, transmucosal administration, and the like. Exemplary topical compositions include liquids, creams, lotions, pastes, sprays, foams, ointments, and the like that include the presently-disclosed compositions. All such compositions are collected referred to herein as “viscous spreadable solutions.”

In some embodiments topical administration includes administering a predetermined amount (e.g., volume) of a composition to the skin of a subject in order to administer the compositions transdermally. For instance, in some embodiments the amount (volume) of the composition administered transdermally is determined by dividing the desired amount (mass) of active agent to be delivered by the concentration of active agent (e.g., iodide) in the composition.

In further embodiments the composition is administered by a device calibrated to administer (dispense) a predetermined amount of a composition. Exemplary administration devices include spray devices that are configured to administer and/or can be tuned to administer a predetermined volume of a composition upon being actuated. In some methods the spray device is configured to and/or can be tuned to administer about 10 μL, to about 10 mL of a composition each time the device is actuated. In this manner, an effective amount of the composition can be administered transdermally by actuating a the spray device one or more times to administer a predetermined amount of a composition. Methods that utilize set-volume topical applicators make it easier to topically administer set dosages of active agent.

C. Methods of Synthesis

The presently-disclosed subject matter also includes methods for making the compositions described herein. In some embodiments the method comprises making a mixture containing surfactant and cosurfactant. In some embodiments the mixture containing surfactant and cosurfactant includes a ratio of surfactant to cosurfactant of about 4:1, about 3:1, about 2:1, about 1:1, about 1:2, about 1:3, about 1:4, about 1:5, about 1:6, about 1:7, about 1:8, or about 1:9. Furthermore, in some embodiments an aqueous solution and oil are added to the surfactant and cosurfactant mixture. In specific embodiments the ratio of oil to surfactant and cosurfactant in the solution is about 10:0, about 9:1, about 8:2, about 7:3, about 6:4, about 5:5, about 4:6, about 3:7, about 2:8, or about 1:9.

The order in which the components are added to the solution varies for different embodiments. For example, in some embodiments oil is added to the solution before the aqueous solution, and in some embodiments the aqueous solution is added to the solution before the oil. Those of ordinary skill will appreciate methods for adding the components to yield a composition that comprises desired characteristics.

### EXAMPLES

The following Examples are intended to show certain embodiments of the presently-disclosed subject matter. They are exemplary of the presently-disclosed subject matter and are not to be construed as being limiting thereof. Thus, the compositions described in the Examples are non-limiting examples of embodied compositions of the presently-disclosed subject matter.

### Example 1

### 1. Materials and Methods

For this example, potassium iodide (KI) (Sigma Aldrich, Mo., USA), Span 20 and Pyrene were purchased from Sigma Aldrich (MO, USA). Denatured alcohol was purchased from Fisher Scientific (Waltham, Mass.). Capryol® 90 and Transcutol®P were obtained from Gattefosse (Lyon, France). De-ionized water was also used in this example.

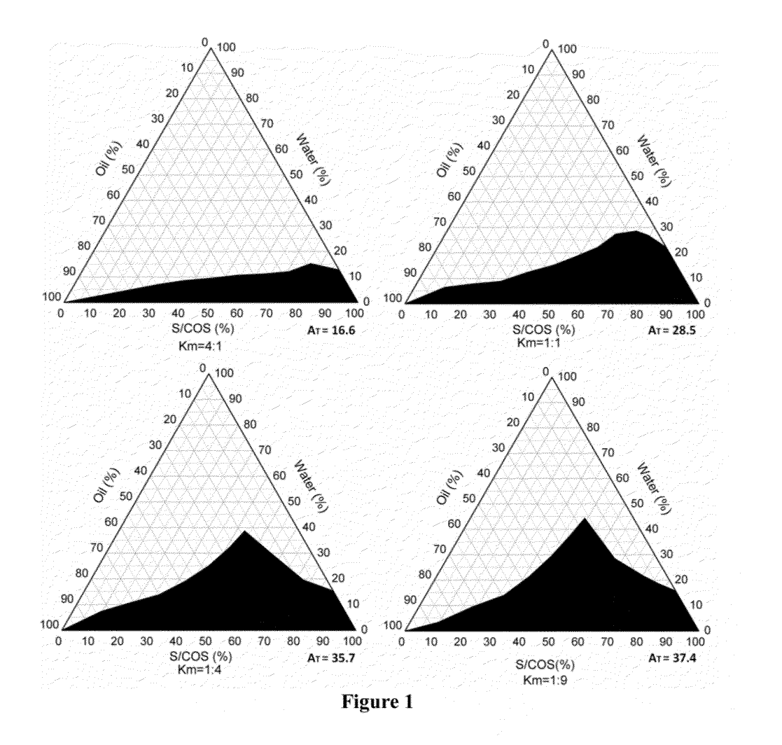

Pseudo-ternary phase diagrams were constructed to evaluate the miscibility of the basic exemplary composition components at 25° C. A series of different ratios (Km) of surfactant (Span 20) to cosurfactant (denatured alcohol) were prepared at 4:1, 1:1, 1:4, and 1:9 then it was followed by the addition of oil (Capryol 90) at different weight ratios of oil to mixture of surfactant and cosurfactant of 10:0, 9:1, 8:2, 7:3, 6:4, 5:5, 4:6, 3:7, 2:8 and 1:9 respectively. Water was titrated drop by drop to the three-component mixture (under constant magnetic stirring) until a transition point where transformation from transparent (optical monophase) to turbid (optical diphase) was reached. A boundary line connecting all transition points was drawn, and the monophasic area A_{T} beneath this boundary line was calculated by using Origin 8 software (OriginLab Corporation, Northhampton, Mass.). The resulting diagrams can be seen in FIG. 1. A_{T} was used to evaluate water solubilization capacity into the oil.

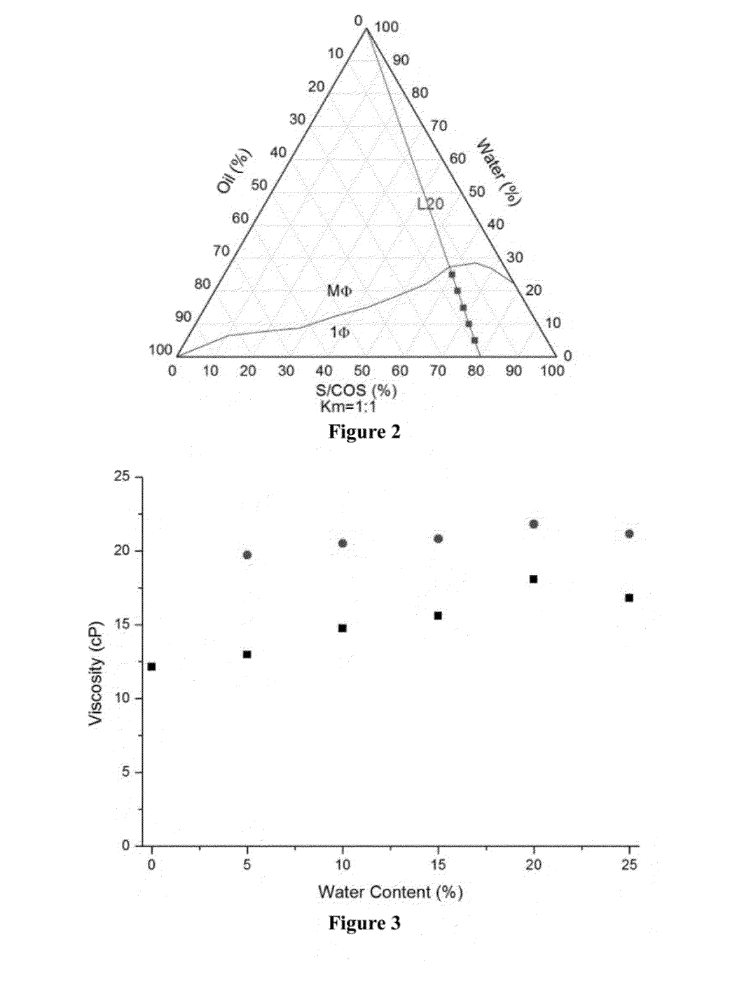

The pseudo-ternary phase diagram at a constant surfactant/cosurfactant ratio Kₘ of 1:1 was chosen for further development because there is sufficient area in pseudo-ternary phase diagram which could form a composition at this ratio while cosurfactant (denatured alcohol) was kept relatively low in the composition. A dilution line (L20) was plotted linking 100% water to a mixture of oil and S/COS of 20% and 80% (FIG. 2). Data points on this line have a constant ratio of oil to S/COS of 1:4. The intersection between the dilution and the boundary lines was recognized as the exemplary composition with maximum water solubilization capacity.

Five selected exemplary compositions (Table 1) with different water contents from L20 were further tested. Blank compositions were first prepared by manually mixing Span 20, denatured alcohol, Capryol 90, and water. Then compositions were incorporated with potassium iodide (KI) at a constant concentration of 50 mg/mL by vortex mixing.

Accelerated Microstructure Stability Testing

Two milliliters of selected exemplary compositions with and without potassium iodide were centrifuged at 13,000 RPM (13,793 g) for 30 minutes using Eppendorf 5415C centrifuge.

Twenty milliliters of selected exemplary compositions were stored in sealed vials at a 40° C. stability chamber for 3 weeks.

Droplet Size Measurements

The mean droplet size of selected exemplary compositions was determined by dynamic light scattering using Zetasizer Nano ZS (Malvern Instruments Inc, Westborough, Mass., USA). Light was scattered at a fixed angle of 90°. Refractive index and viscosity values were inputted into the program to determine the mean droplet size accurately. All measurements were obtained at 25° C. Triplicate measurements were taken.

pH Measurements

The pH values of selected exemplary compositions were acquired using Orion 520A pH meter (Thermo Fisher Scientific Inc, Pittsburgh, Pa., USA). The pH probe was inserted into 20 mL of liquids and values were recorded when the reading stabilized. All measurements were done in triplicate.

Viscosity Measurements

The kinematic viscosities of the selected exemplary compositions were determined using Cannon-Fenske routine viscometer (Cannon Instrument Company, State College, Pa., USA) at ambient temperature. Kinematic viscosity was obtained by multiplying efflux time of sample flowing through the capillary tube of the viscometer by the viscometer constant. Thereafter, the dynamic viscosity was determined by multiplying the value of kinematic viscosity by the sample density. Triplicate measurements were performed.

Conductivity Measurements

Conductivity measurements were performed using conductivity meter FE30/FG3 (Mettler-Toledo Inc, Columbus, Ohio, USA) at 25° C. Conductivity diagram was obtained through drop by drop water titration to the mixture of oil and surfactant/cosurfactant (S/COS) at a constant ratio of 1:4 in a beaker. The conductivity sensor was soaked in the liquid and the reading was recorded when the signal indicating the endpoint was achieved. The conductivity of the five selected exemplary compositions after the incorporation of KI was carried out using the same methodology. All measurements were carried out in triplicate.

Differential Scanning Calorimetry (DSC)

The thermo behavior of selected exemplary compositions were explored by DSC scanning using a DSC Q2000 (TA instrument, DE, USA) equipped with a nitrogen cooler. Composition samples (approximately 6 mg) were weighted and placed into Tzero aluminum pans and sealed with Tzero hermetic lids using a Tzero sample press. The samples were initially equilibrated at −90° C. for 3 minutes, and then went a heating process at a constant heating rate of 5° C./min. Results were analyzed using universal analysis 2000 data analysis software.

In Vitro Permeation Studies

Human skin samples (chest and abdominal regions) were purchased from National Disease Research Interchange (NDRI, Philadelphia, Pa., USA). Subcutaneous fatty tissues were removed from skin using a lancet after soaking the skin in a 60° C. water bath for 1 minute. Thereafter, skin samples were washed with di-ionized water. Prior to the actual permeation study, the fat free skin was stored refrigerated at 4° C.

To investigate KI composition diffusion through the skin, Franz cells (PermeGear Inc, Hellertown, Pa., USA) were utilized. The receptor volume of each cell was 5 mL and the diffusion area was 0.64 cm². Prior to mounting the skin samples, each receptor was filled with 5 mL di-ionized water. Successively the skin samples were clamped in between the receptor (down) and donor (up) holding the stratum corneum side up. Prior to the experiment, the jacketed receptor was kept for 1 hour at 37° C. using a water bath with magnetic stirring. Afterwards, 1 mL of each selected exemplary composition with 50 mg/mL KI was loaded to the donor compartment and each donor cell was sealed with Parafilm® to avoid the evaporation of composition components. A KI solution (1 mL of 50 mg/mL solution) was used as the control. Then, 250 μL of the aqueous liquid were withdrawn from the sampling port of receptor and diluted with di-ionized water to 5 mL at different time points (0 h, 2 h, 4 h, 6 h, 8 h, 12 h, and 24 h). Simultaneously, equal volume of di-ionized water was replaced into the liquid. The diluted samples were filtered through 0.45 μm Millex® filter (Millipore, Billerica, Mass.) and analyzed using Orion iodide selective electrode (Thermo Fisher Scientific Inc, Pittsburgh, Pa., USA). Three replicates were carried out for each selected exemplary composition.

The concentration of iodide in the receptor at every time point was calculated after incorporating the dilution factor. Then the cumulative amount of KI permeated across the skin per unit area (mg/cm²) was obtained by using the following equation:

$${Q}_{n} = \frac{{C}_{n} \times {V}_{0} + {\sum }_{i = 1}^{n - 1} {C}_{1} \times {V}_{1}}{S}$$

where Cₙ is the undiluted sample concentration (liquid concentration in the receptor) at n^{th} sampling time point, Cᵢ is undiluted sample concentration (liquid concentration in the receptor) i^{th} sampling time point, Vₒ is the receptor volume (5 mL), Vᵢ is the sampling volume (250 μL), and S is diffusion area (0.64 cm₂). All Qₙ values at each time point were plotted as a function of time. Thus, the steady state flux (Jₛₛ mg/cm² per hour) was calculated for every exemplary composition. Jₛₛ is the slope of linear portion of cumulative iodide amounts.

### 2. Results and Discussion

Accelerated Microstructure Stability Testing

Centrifugal forces accelerate physical instability of compositions and lead to turbidity and phase separation. Brownian motion maintains droplets' kinetic energy which causes irregular movements of small droplets, so it prevents droplet settling. Additionally low interfacial tension and droplets kinetic energy lead to inhibition of creaming, sedimentation, flocculation, and coalescence.

All selected exemplary compositions of the presently-disclosed subject matter (B-F) in Table 1, below, in the presence and the absence of KI had no phase separation by the end of 30 minutes under high centrifugal forces which is a sign of the strong physical stability of compositions.

TABLE 1
Composition, pH and Z-average diameter at 25° C.

| | Water/Span20/ | | | |
|---|---|---|---|---|
| | Denatured Ethanol/ | | | |
| | Capryol90 ® | | | Z-average |
| | composition | KI | | diameter |
| Composition | (w/w %) | (g/mL) | pH | (nm) |
| Example A | 5/38/38/19 | None | 6.53 ± 0.01 | 0.51 ± 0.03 |
| Example B | 10/36/36/18 | None | 6.50 ± 0.00 | 0.71 ± 0.03 |
| Example C | 15/34/34/17 | None | 6.30 ± 0.01 | 1.04 ± 0.02 |
| Example D | 20/32/32/16 | None | 6.09 ± 0.01 | 1.68 ± 0.05 |
| Example E | 25/30/30/15 | None | 5.93 ± 0.01 | 4.86 ± 0.05 |
| Example F | 5/38/38/19 | 0.05 | 5.66 ± 0.02 | Size under |
| | | | | detection |
| | | | | limit |
| Example G | 10/36/36/18 | 0.05 | 5.62 ± 0.02 | 0.45 ± 0.03 |
| Example H | 15/34/34/17 | 0.05 | 5.50 ± 0.01 | 0.71 ± 0.03 |
| Example I | 20/32/32/16 | 0.05 | 5.47 ± 0.01 | 1.09 ± 0.03 |
| Example J | 25/30/30/15 | 0.05 | 5.43 ± 0.01 | 2.30 ± 0.04 |

Thermal stability testing under 40° C. showed no turbidity by the end of three weeks, which is the evidence that embodiments of compositions are of good physical stability at temperature close to body temperature.

Droplet Size Measurements

Dynamic light scattering technique measures droplet size through direct measurement of the droplet diffusion coefficient in a dispersed medium undergoing Brownian motion then the droplet size is obtained from the Stokes-Einstein equation. In the selected exemplary compositions, surfactant, cosurfactant, and oil form the external phase, while water (aqueous core) is the internal phase. The existing boundaries between oil and aqueous core are composed of the polar parts of Span 20, the water and denatured alcohol.

The results for droplet size are shown in Table 1. All exemplary compositions had small droplet size distributions which indicate the system is stable. It can be deduced in these exemplary w/o composition systems that the apparent droplet sizes had increased dramatically as the water content increased. Without being bound by theory or mechanism, this can be attributed to the increase of the amount of water molecules in the aqueous core.

Surprisingly, the addition of KI to the exemplary compositions shrinks water droplets. Without being bound by theory or mechanism, this is due to the salting-in effect which occurs between inorganic anionic ions such as iodide ions and water molecules. Thus, anionic ion makes water less polar and makes the organic components dissolve more readily into internal aqueous clusters. A schematic demonstration of exemplary compositions with and without KI is shown in FIG. 3. Iodide, a member of Hofmeister ion series, tends to increase the solubility of nonpolar components in aqueous solvent by decreasing the surface tension between water and organic molecules. Thus, the polar region (water content in the core) diminishes and the existing boundary between polar and non-polar molecules shrinks consequently, which causes the droplet size to decrease.

pH Measurements

pH decreased from 5.66 to 5.43 when the amount of water increased from 5% to 25% (see Table 1). Without being bound by theory or mechanism, higher water content tends to increase the ionization of organic components such as the hydrophilic portion of Span 20 which is very weak acidic. This can provides extra protons to decrease the pH value. The incorporation of KI further enhances organic component solubilization in water which results in extra free protons and lower pH. The pH values of all selected exemplary compositions are physiologically acceptable for topical uses.

Viscosity Measurements

Viscosity of exemplary emulsion compositions are a polynomial function which depends on concentrations of water, surfactant, cosurfactant, and oil in composition. Black dots in FIG. 2 shows the dynamic viscosity values obtained for all tested compositions without KI as a function of water content. The values were relatively low and ranged between 12 to 22 cP. For the aforementioned compositions, an initial increase of composition viscosity was observed as water content increased from 5 to 20%. Thereafter, a decrease in viscosity was observed as water content increased to 25%.

The observed increase in the viscosity with the increase of water content up to 20% can be explained by the increase of the dispersant phase droplet's volume and surface morphology and the increase in the frequency of collisions between the water droplets. After reaching 20% water content, a viscosity decrease was observed. Without being bound by theory or mechanism, this is probably due to transition of the composition from w/o (spherical dispersed phase) into bicontinuous (non-spherical dispersed phase). When the system exists as a biocontinous phase, the attractive forces between the aggregated water phases become weaker as compared to the w/o composition system and the opportunity to form clusters diminishes.

On the other hand, the viscosity of the selected exemplary compositions after the addition of KI increased to a higher ranger from 19.7 to 21.8 cP. This viscosity increase is probably due to the increase in the formation of transition clusters where iodide makes water more hydrophobic and more free to move. Moreover, it should be noted that the addition of KI may also modify the size and curvature of water droplets.

Conductivity Measurements

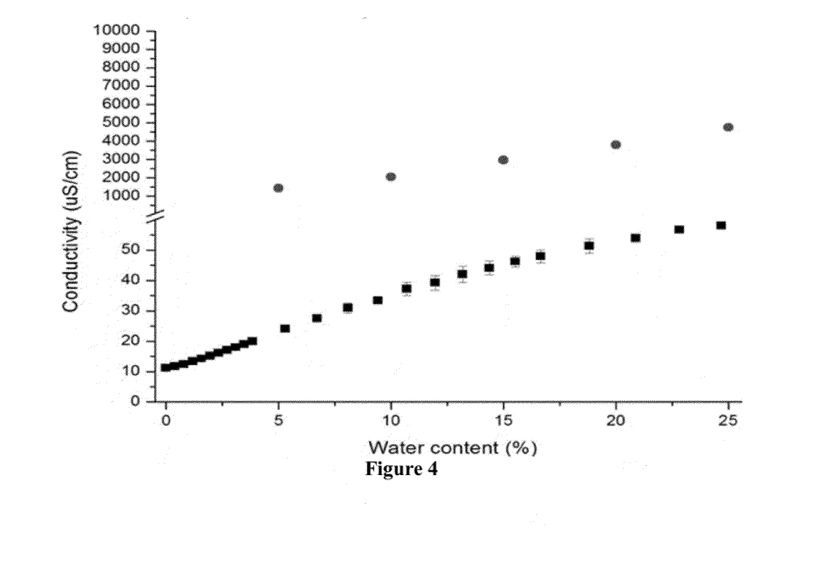

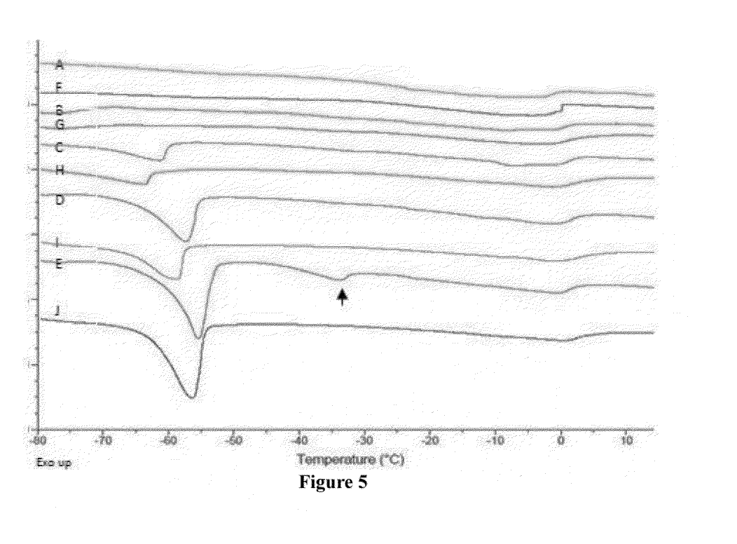

The influence of water content on conductivities of exemplary compositions was presented in FIG. 4. The main graph in FIG. 4 relates conductivity to water content from 0 to 25.5% while the smaller secondary graph at the upper left corner is an expansion of initial conductivity values of blank exemplary composition when the water content increased up to 8%.

In general, when water molecules are dispersed in an oil phase at a small volume fraction, droplets are separated from each other and exhibits minimum interactions and liquid conductivity is low. This causes subtle influence to conductivity. The continuous addition of water increases total number of aqueous droplets which can increase the formation and deformation dynamics of the transient clusters (aggregation of water droplets).

This clusters formation and deformation process is described in three steps: fusion, mass transfer and fission. Transient clusters have significant influence on increasing the electronic conductivity. The transient collisions of water droplets can provide water channels where ions hop from one droplet to the other.

In this system, the percolation threshold exists at around 1% which links the critical water content to conductivity. The critical water content is the point which relates two conductivity behaviors within this system. Below this point, the conductivity increases slowly because of the lower formation frequency of transient clusters while above this point, more transient clusters are formed consequently causing a rapid increase of the conductivity.

In addition, FIG. 4 indicates a rapid increase in conductivity between 1 and 20% water content. This observation is consistent with similar conductivity behavior in exemplary w/o composition systems. On the other hand, above 20% water content, conductivity begins to show some retardation with further water content. The change of conductivity with water content around 20% indicates that the exemplary composition transits from w/o type to bicontinuous type, and the dispersed droplet shape transforms into more non-spherical shape.

The addition of KI ions into the exemplary compositions enhanced conductivities up to 40-folds as compared to exemplary compositions without KI. This observation is consistent with the quantitative charge fluctuation model where the aqueous channels created by the transient clusters contain more dissociated ions which are able to facilitate the overall conductivity tremendously.

The addition of KI ions into selected exemplary compositions at 5, 10, 15, 20, and 25% water content has shown a linear relationship between conductivity and water content which may imply that all these compositions are w/o compositions.

The exemplary compositions' microstructure with 25% water content with and without KI obtained from viscosity and conductivity experiments were consistent. Without being bound by theory or mechanism, it is likely that a bicontinuous (non-spherical) type exists in no KI systems as compared to w/o (spherical) type in KI systems. The addition of KI changes the interface curvature and makes the droplets more spherical.

Differential Scanning Calorimetry (DSC)

Thermo behavior can be used to explore component interactions and the microstructure of compositions. Ethanol has no phase change in the DSC scanning range because its melting point is below −100° C. All curves showed gradual inclination when temperature ranged from −40° C. to 0° C. then followed by a small endothermic peak which is related to Span 20 microstructure where the packing of surfactant long carbon chains into an ordered crystalline structure is very difficult as a result these carbon atoms could align in various configurations.

As water content increases to 15%, the amount of Span 20, denatured alcohol, and Capryol 90 decrease proportionally and an endothermic peak was observed at approximately −60° C. This peak became larger and sharper when water percentage had risen up to a higher level and its location shifted to a higher temperature range. This particular peak represents the melting point of Capryol 90. Capryol 90 is bounded with surfactants and cosurfactants and especially at high level of cosurfactant contents (denatured alcohol molecules) which interact well with Capryol 90 to keep it in a non-freezing state (liquid phase) with no phase changes during the cooling and heating process. When the denatured alcohol content decreased, melting and solidification of Capryol 90 was observed because of less interaction between both. Consequently, when denatured alcohol and Span 20 contents further decreased, less influence was observed on the melting point of Capryol 90 and shifted to a higher temperature. The interaction between composition components (exemplary compositions H, I, and J) was influenced by the addition of KI which slightly lowered the melting point of Capryol 90.

There was a broadened endothermic peak in the DSC curve for exemplary composition E with 25% water content (see the arrow), which is attributed to melting of “bound” water in the core of aqueous domain. In w/o composition, water droplets have very strong interactions with surfactant and cosurfactant due to hydrogen bonding. Thus, when water content is low, most water molecules are located in the interfacial layer which cannot be solidified and will be melted during DSC cycle. As water content increased in exemplary w/o compositions, more water molecules accumulate in the aqueous core where the interaction forces with organic components are relatively less strong compared to water molecules at the interface. So water molecules in the aqueous core undergo solidification and melting in this DSC cycle.

The broadness of this peak may be attributed to water molecules at different locations with different interaction forces. However, this peak disappears when 25% water content composition was loaded with KI (exemplary composition J). Without being bound by theory or mechanism, the addition of KI suggested that water molecules become more hydrophobic and trigger more interaction between them and other organic components, which is consistent with previous pH, droplet size and viscosity results.

In vitro iodide composition permeation studies

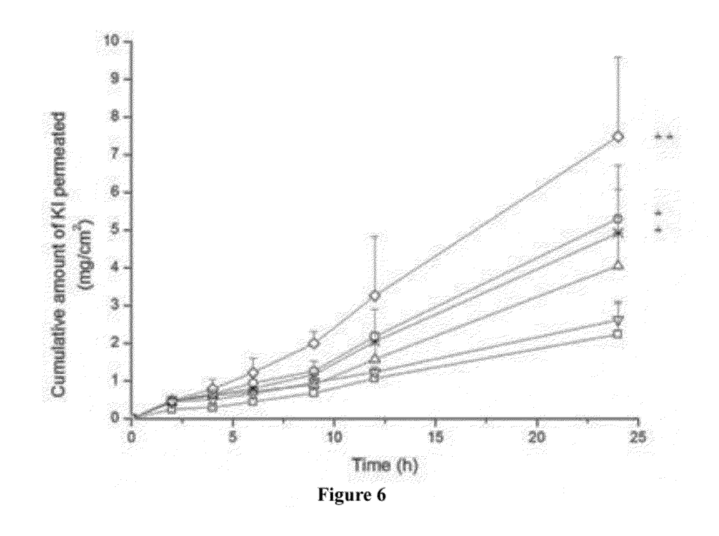

Cumulative amount of KI permeated through human skin over 24 hours for selected exemplary compositions is depicted in FIG. 6. Results indicated that at the end of 24 hours exemplary KI compositions with 5 and 10% water content did not significantly show better iodide permeation through skin as compared to the control sample (KI solution; P>0.05). However, exemplary KI compositions with 15, 20, and 25% water content exhibited significant iodide permeation through the skin (student t-test, 15 and 20% P<0.05, and 25% P<0.02).

Cumulative amount of permeated KI of exemplary composition H and I (15 and 20% water content) have a magnitude 2 times as great as it of KI solutions, and additionally cumulative amount of permeated KI has a further enhancement for exemplary composition J (7.48±2.11 mg/cm²). Values of flux at steady-state and cumulative permeated KI were listed in Table 2, below.

TABLE 2
Cumulative Permeated KI (Q₂₄) and Flux at steady-state
(Jₛₛ) (mg/cm²/h) of selected exemplary compositions.

| Composition | Cumulative Permeated KI | Flux at steady-state (Jₛₛ) |
|---|---|---|
| (0.05 g/mL KI) | (Q₂₄) (mg/cm²) | (mg/cm²/h) |
| Solution | 2.23 ± 0.88 | 0.101 ± 0.039 |
| Example F | 2.62 ± 0.43 | 0.113 ± 0.026 |
| Example G | 4.05 ± 1.30 | 0.209 ± 0.073 |
| Example H | 4.92 ± 1.16 | 0.247 ± 0.087 |
| Example I | 5.30 ± 1.40 | 0.267 ± 0.074 |
| Example J | 7.48 ± 2.11 | 0.331 ± 0.073 |

Flux at steady-state (Jₛₛ) indicates that when water content increases from 5 to 25%, iodide permeates through skin at a higher rate. These results indicate that organic components (Span 20, Capryol 90, and denatured alcohol) in the exemplary compositions act as penetration enhancers. They potentially modify the lipid structure within the stratum corneum and make it looser and more porous for iodide permeation. In addition, more addition of water in w/o composition could influence iodide permeability to a higher degree. One possible reason to explain this observation is that skin is hydrated by water and thus undergone swelling up, which results in producing more void spaces in skin as wider diffusion channels for iodide. Thus, permeation profile of iodide enhancement derived by embodied compositions is a combination of factors including permeation enhancer and skin hydration.

### Example 2

### 1. Materials and Methods

Unless otherwise stated, the same materials and testing methods as those described in Example 1 were implemented for Example 2. One difference was the use of anhydrous ethanol (Thermo Fisher Scientific Inc.) in Example 2, rather than denatured alcohol.

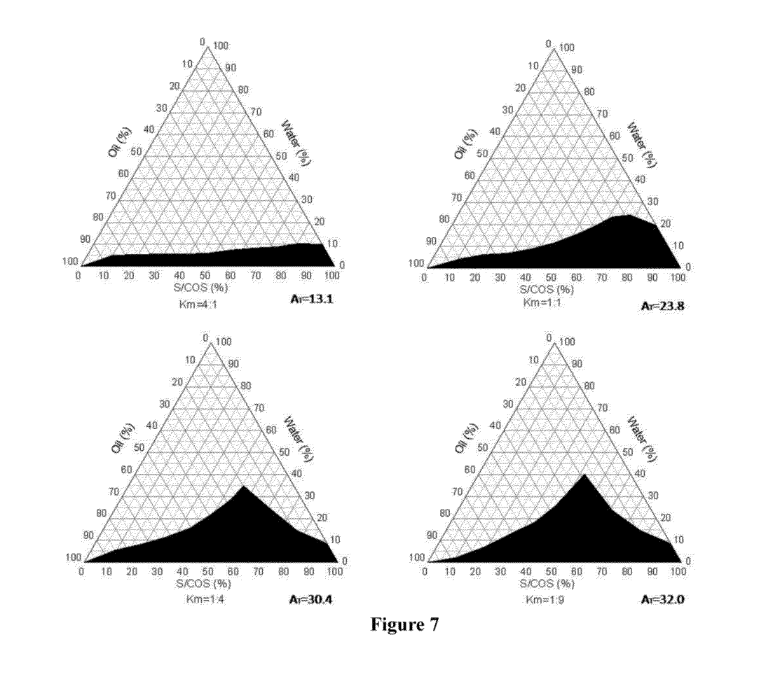

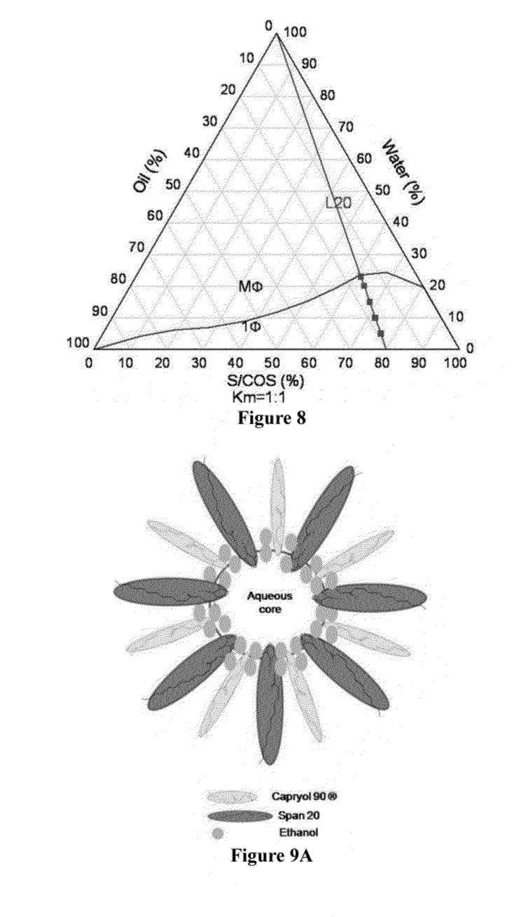

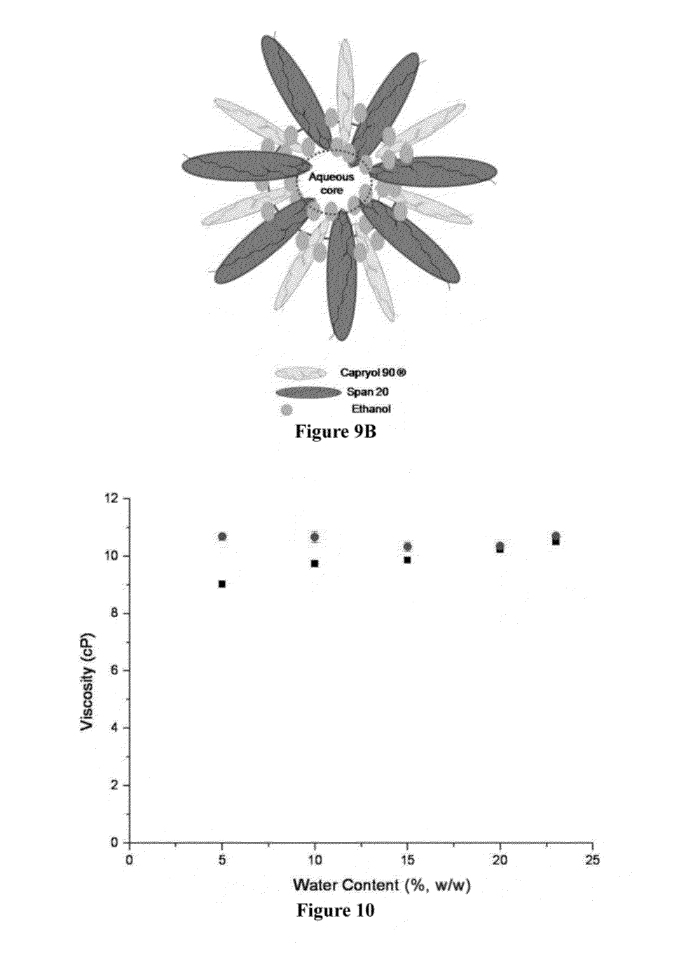

Pseudo-ternary phase diagrams were also constructed and can be seen in FIG. 7. Furthermore, the pseudo-ternary phase diagram at a constant surfactant/cosurfactant ratio Kₘ of 1:1 having a dilution line (L20) plotted linking 100% water to a mixture of oil and S/COS of 20% and 80% is shown in FIG. 8. Data points on this line have a constant ratio of oil to S/COS of 1:4. The intersection between the dilution and the boundary lines was recognized as the composition with maximum water solubilization capacity. The following solubilization capacities A_{T} were calculated at different surfactant (S): cosurfactant (COS) ratio Kₘ (Kₘ:4:1, A_{T}: 16.6, Kₘ:1:1, A_{T}:28.5, Kₘ:1:4, A_{T}:35.7, Kₘ:1:9, A_{T}:37.4).

Five selected exemplary compositions with different water contents from L20 that were further tested are shown in Table 3.

TABLE 3
Composition, pH, and Z-average diameter at 25° C.

| | Water/Span20/ | | | |
|---|---|---|---|---|
| | Ethanol/ | | | |
| | Capryol 90 ® | | | Z-average |
| | composition | KI | | diameter |
| Composition | (w/w %) | (g/mL) | pH | (nm) |
| Example A | 5/38/38/19 | None | 5.20 ± 0.01 | Size under |
| | | | | detection limit |
| Example B | 10/36/36/18 | None | 5.13 ± 0.01 | 1.48 ± 0.03 |
| Example C | 15/34/34/17 | None | 5.00 ± 0.01 | 2.48 ± 0.39 |
| Example D | 20/32/32/16 | None | 4.94 ± 0.01 | 4.36 ± 0.04 |
| Example E | 23/30.8/30.8/15.4 | None | 4.82 ± 0.01 | 5.57 ± 0.33 |
| Example F | 5/38/38/19 | 0.05 | 5.68 ± 0.00 | Size under |
| | | | | detection limit |
| Example G | 10/36/36/18 | 0.05 | 5.60 ± 0.01 | 1.07 ± 0.06 |
| Example H | 15/34/34/17 | 0.05 | 5.40 ± 0.01 | 2.19 ± 0.15 |
| Example I | 20/32/32/16 | 0.05 | 5.38 ± 0.01 | 2.88 ± 0.21 |
| Example J | 23/30.8/30.8/15.4 | 0.05 | 5.32 ± 0.01 | 4.51 ± 0.15 |

As shown in FIG. 7, A_{T} represents the monophasic area (solubilization capacity) and it tends to increase as the amount of span to ethanol increases in the exemplary compositions (Kₘ: 4:1, A_{T}: 13.1; Kₘ: 1:1, A_{T}: 23.8; Kₘ: 1:4, A_{T}: 30.4; Kₘ: 1:9, A_{T}: 32.0).

Droplet size and pH measurements

The results for droplet size are depicted in Table 3. Mean droplet size was not measurable for exemplary compositions containing 5% water by dynamic light scattering. Without being bound by theory or mechanism, these compositions lations may resemble cosolvent systems. It is possible that when water content in the system is low, water molecules can stay separate without the forming an aqueous droplets. As the water content increases to 10%, the average droplet size becomes more than 1 nm. It was observed that as water content further increased, droplet size of the compositions also increased.

The pH values of all selected exemplary compositions were physiologically acceptable for topical uses. It was observed that the pH decreased from 5.20 to 4.82 when the amount of water increased from 5% to 23% (Table 3). However, after the incorporation of KI, pH values increased slightly from 5.68 to 5.32 (5% to 23% water content).

Viscosity Measurements

FIG. 10 depicts the dynamic viscosity values obtained for all tested exemplary compositions in the presence and absence of KI as it relates to water contents. The viscosity values for exemplary compositions without KI were relatively low and ranged between 9 to 11 cPoise. It was observed for the aforementioned compositions, viscosity increased as the water content increased from 5 to 23%. The observed increase in the viscosity with the water content is dependent upon the increase of the dispersant phase droplet's volume and the increase in the frequency of collisions between the water droplets in w/o composition systems.

It was noted that after the addition of KI the viscosity of the exemplary compositions (FIG. 10) increased slightly (10 to 11 cPoise). The increase of viscosity is not only due to more dense internal aqueous phase, but also due to the increase in the formation of aqueous transition clusters where iodide ions cause aqueous phase to become more hydrophobic and free to move (37).

Conductivity Measurements

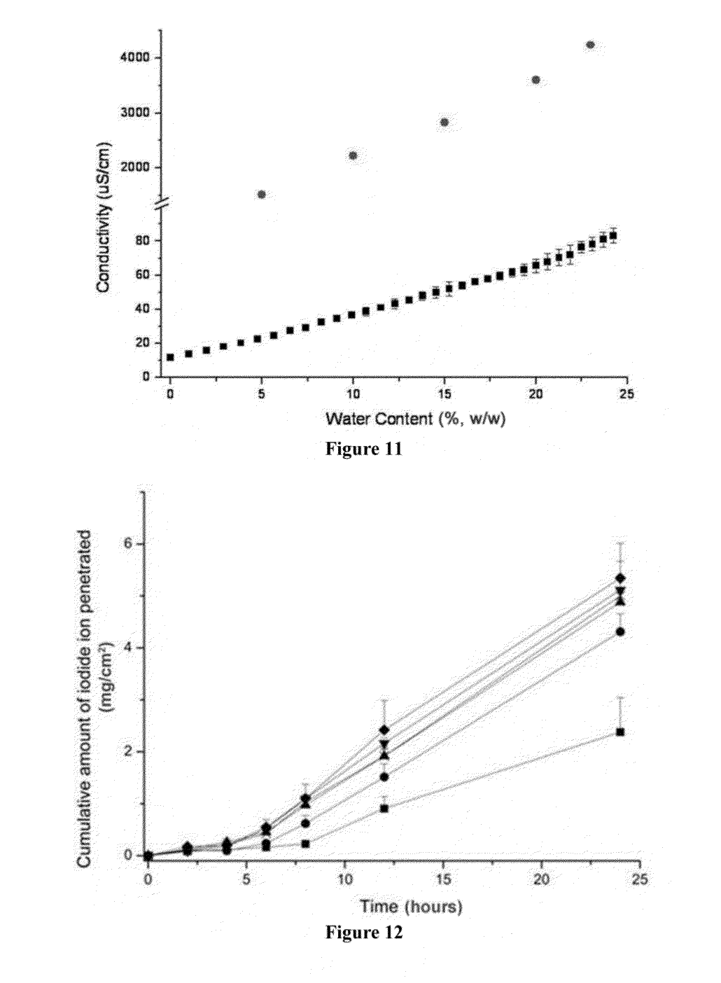

The influence of water content on conductivities of selected exemplary compositions is presented in FIG. 11. The main graph relates overall ions conductivity of water content from 0 to 24.5%. Addition of KI into the selected exemplary compositions enhanced conductivities more than 50-fold as compared to exemplary compositions without KI.

In Vitro Composition Skin Permeation Studies

Cumulative amounts of iodide that permeated through human skin over 24 hours for selected exemplary compositions are depicted in FIG. 12. Results indicated that at the end of 24 hours all exemplary KI compositions with different water contents (5%, 10%, 15%, 20%, and 23%) had significantly better iodide permeation through skin as compared to the control sample (KI solution; student paired t-test, P<0.05).

Exemplary composition F exhibited the lowest cumulative amount of iodide that permeated the skin at the end of 24 hours out of the five selected exemplary compositions (F to J). It permeated about 2 times the amount of KI as compared to the solution after 24 hours. On the other hand, exemplary composition J was the most effective composition for iodide permeation study (about 2.5 times of KI solutions) after 24 hours. Statistically, iodide permeation at the end of 24 hour for exemplary composition J (23% water content) was significantly higher (student paired t test, P<0.05) as compared to exemplary composition F (5% water content), but had no significant difference compared to exemplary composition G (10% water content), H (15% water content), and I (20% water content) (student paired t test, P>0.05).

Values of steady-state flux and cumulative amounts of iodide that permeated are listed in Table 4. Steady-state flux values (Jₛₛ) indicates that selected exemplary compositions (F to J) had a significant better permeation of iodide compared to KI solution. Likewise, larger amounts of water in exemplary w/o compositions could influence iodide permeability to a higher extent since exemplary composition J (23% water content) had the highest flux rate 0.266±0.037 mg/cm²/h. In the presence of water, skin is hydrated and exists in a swollen state, thus more void spaces within the skin create wider diffusion channels (42). In summary, the permeation profile of iodide within compositions is affected by a combination of factors including permeation enhancement and skin hydration.

TABLE 4
Cumulative Permeated KI (Q₂₄) and Flux at steady-state
(Jₛₛ) (mg/cm²/h) of selected compositions

| Compositions | Cumulative permeated iodide | Flux at steady-state (Jₛₛ) |
|---|---|---|
| (50 mg/mL KI) | (Q₂₄) (mg/cm²) | (mg/cm²/h) |
| Solution | 2.38 ± 0.66 | 0.127 ± 0.036 |
| Example F | 4.31 ± 0.34 | 0.228 ± 0.014 |
| Example G | 5.00 ± 0.66 | 0.252 ± 0.038 |
| Example H | 5.11 ± 0.29 | 0.254 ± 0.014 |
| Example I | 4.88 ± 0.50 | 0.245 ± 0.029 |
| Example J | 5.35 ± 0.53 | 0.266 ± 0.037 |

Accelerated Microstructure Stability Testing

Twenty milliliters of selected exemplary compositions were stored in sealed vials at a 40° C. stability chamber for 4 weeks. Three replicates were carried out for each composition.

All selected exemplary compositions (A-J) in Table 3 in the presence and the absence of KI had no phase separation and clarity change by the end of 30 minutes under high centrifugal forces (13,000 rpm, 13,793 g) which is a sign of the strong physical stability of the compositions.

Thermal stability testing under 40° C. showed no turbidity by the end of three weeks, thus lending further support to the physical stability of the composition under thermal stress.

Chemical Stability

Potassium iodide-starch test paper was utilized to test the existence of iodine. Four standard iodine solutions (0.025 mg/mL, 0.25 mg/mL, 0.5 mg/mL, 1 mg/mL) were prepared by dissolving iodine in ethanol/water (50/50 w/w) solution. These standards were utilized as indicators for any presence of iodine in the compositions. Exemplary composition J (23% water composition with 50 mg/mL KI) was selected to observe the presence of iodine as a function of time (0 week, 2 weeks, and 4 weeks) at ambient temperature. The test paper was dipped into the selected exemplary composition for 30 seconds, followed by washing with a small volume of water to provide the aqueous environment for the triiodide starch reaction.

Thus, chemical stability was performed by exploring the presence of degradation product iodine, as iodine reacts with starch in the presence of iodide and expresses blue-black color. A series of iodine solution standards with different concentrations (FIG. 7) were prepared at 0.05% (2.5E-2 mg/mL iodine), 0.5% (2.5E-1 mg/mL iodine), 1% (5E-1 mg/mL iodine), and 2% (1 mg/mL iodine). The rational for preparing these four iodine standards was related to the assumption that if the percentages of degradation of the initial concentration of potassium iodide in compositions (50 mg/mL) were 0.05%, 0.5%, 1% and 2%, then the concentrations of iodine that should be formed will be 2.5E-2, 2.5E-1, 5E-1 and 1 mg/mL respectively.

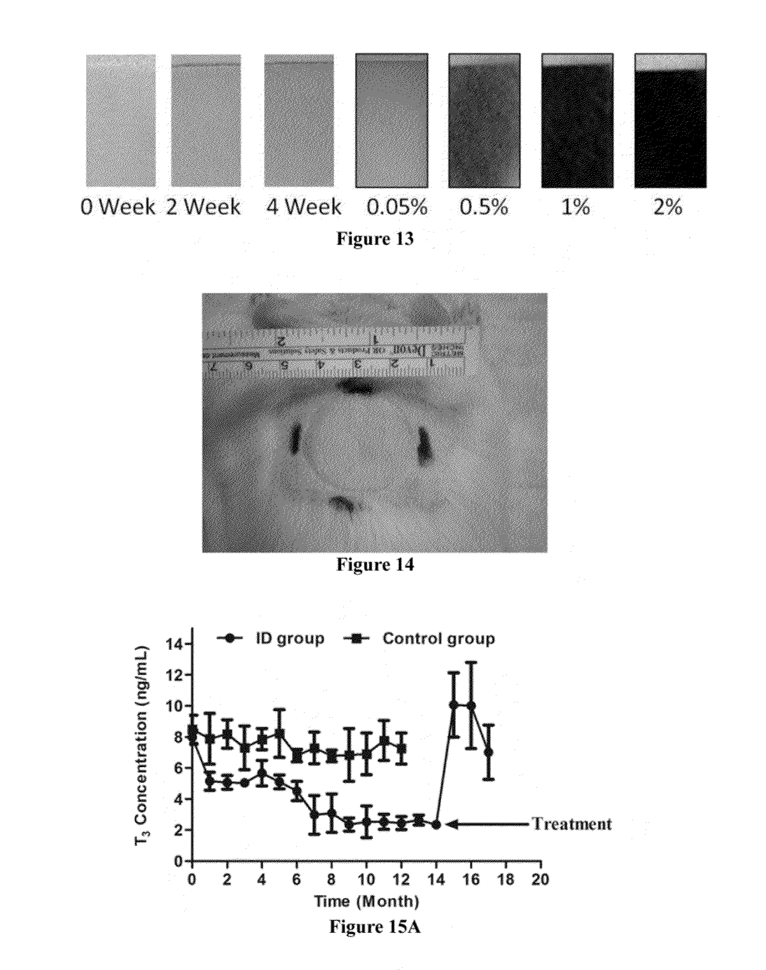

With increasing iodine concentration the blue-black color became more intense. Samples were collected over 1 month period (FIG. 13). At the end of one month, the absence of blue-dark color in iodide-starch test paper indicates the percentage of degradation product iodine is much less than 0.5%. Thus, the exemplary KI composition with 23% water was chemically stable for at least one month.

### Example 3

In this Example an embodiment of the present compositions was administered in vivo to evaluate its applicability and efficiency for treating iodide deficiency. Iodine deficiency was monitored through evaluating the levels of T3, T4, and TSH, iodide concentration in urine, thyroid gland size, and body weight. In particular, levels of T3, T4, and TSH in the bloodstream correlate with the amount of ingested iodide, and urinary iodide concentration is a biomarker for estimating the amount of iodide available in the blood.

### Materials

Potassium iodides (KI), Span 20, Sodium carbonate, Sodium bicarbonate, and HPLC grade methanol were purchased from Sigma Aldrich Anhydrous ethanol was obtained from Fisher Scientific (Thermo Fisher Scientific Inc.). Capryol® 90 was purchased from Gattefosse. Sodium hydroxide and Alcohol reagent were obtained from Fisher Scientific. Millipore water was used in this Example.

Preparation of Composition

The same procedure followed in Examples 2 and 3 was utilized to prepare the iodide microemulsin (50 mg/mL) compositions. In brief, microemulsion (10 mL) was prepared by mixing (3.080 mL) Span 20, (3.080 mL) ethanol and (1.540 mL) capryol 90 as the surfactant, cosolvent and the oil phase, respectively. Water (2.3 mL) was then added (under continuous stirring) until a transparent mixture (optical monophasic) was obtained. Afterwards, KI was added to obtain a concentration of 50 mg/ml and the mixture was sonicated until all KI crystals were dissolved.

Animals

5-6 week-old male Sprague-Dawley rats, (n=10) weighing 150-200 g, were used for this Example. Rats were obtained from Harlan Laboratory (Indianapolis, Ind.). Upon arrival all rats were housed in cages.

The rats were divided randomly into two groups according to their diet type. The first group was fed iodine deficient rodent diet 93G (Harlan Laboratories) that contained 20 μg/kg iodide, and the control group received AIN-93G purified diet (Harlan Laboratories) that contained 200 μg/kg iodide. Tap Water was tested for iodine content (<5 μg/L) and was freely available.

The iodine deficient (ID) group (n=5) was treated weekly after month 14 with 7 mg of KI. Specifically, the iodide microemulsion composition was administered transdermally onto a shaved area at the back of the neck of each rat (FIG. 14). The treatment surface area was limited to specified size of 7±0.5 cm². The iodide microemulsion composition was administered using a constant volume topical actuator (Aptar Pharma, Congers, N.Y.) that delivered 140±2 μL (50±1 μg/μL of KI) of the microemulsion with each actuation (140 μl contains 7 mg KI).

Animals were moved biweekly to metabolism cages in order to collect plasma and urine samples. Blood samples were taken from the tail vein under anesthesia. Each blood sample (0.5 mL) was placed in EDTA tubes and centrifuged to separate the plasma. Plasma samples were stored at −80° C. for future analysis. Body weights were monitored and recorded biweekly throughout the experiment for both groups. At the end of treatment, animals were anesthetized and sacrificed, and thyroid glands were surgically removed and evaluated.

T3, T4, and TSH Levels

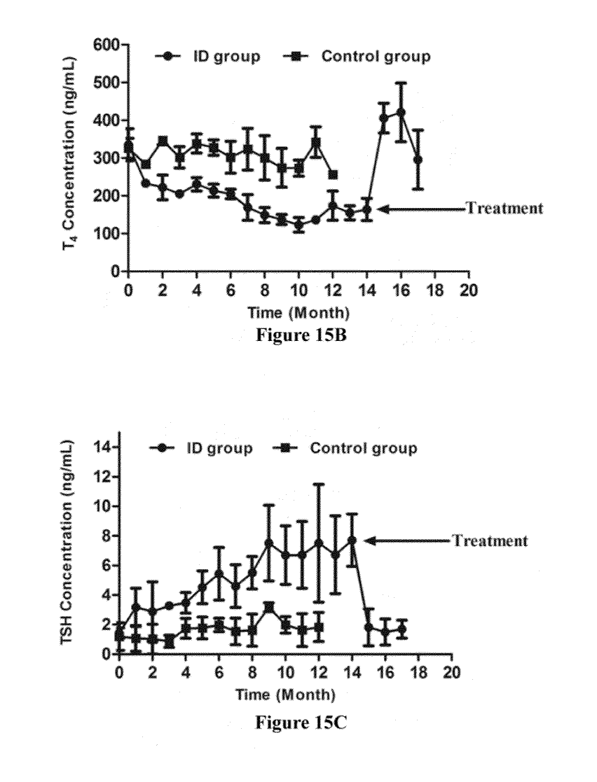

During an early monitoring phase, iodine deficiency was induced in rats to determine the values of T3, T4, and TSH, and the patterns which reflect this deficiency. The plasma levels of T3, T4, and TSH in iodine deficient rats and in control group were monitored over a 17 month period, and the results are shown in FIGS. 15A to 15C.

Thyroid stimulating hormone (TSH) and Thyroid hormones (T3 and T4) were measured in plasma using BioPlex® (Bio-Rad, Hercules, Calif.) using rat thyroid hormone immunobead assay MILLIPLEX® MAP kit. The assay procedures were performed as described by the kit's protocol (Millipore). In brief, plasma samples were diluted (1:5) with assay buffer. Thereafter, 25 μL of sample were incubated with antibody-coated magnetic beads overnight at 4° C. The following day all the beads were washed 3 times using an automated magnetic plate washer Tecan HYDROFLEX™ (Tecan Group Ltd., Mannedorf, Switzerland) and further incubated with detection antibody overnight. After incubation, streptavidin-phycoerythrin solution was added per well. Standard calibration curves for known concentrations of T3, T4, and TSH were constructed to convert the fluorescence units to concentration values (ng/ml). All standards, quality controls, and plasma samples were run in duplicate.

At the beginning of the monitoring period, the thyroid hormones T3, T4, and TSH showed similar levels in both groups. At the end of the first month, there was a statistically significant reduction in T3 and T4 levels in ID group as compared to the control group (P<0.001). The values of T3 and T4 decreased up to 5.2±0.6 ng/ml and 233±9 ng/ml in ID group as compared to 7.9±1.6 ng and 283±9 in the control group. Thereafter, no statistically significant reduction in T3 and T4 levels was noticed up to 5 months (P>0.1). After month 5, there was a further reduction in T3 and T4 in ID diet until a minimum plateau was reached at month 7 for T3 and continued slow reduction in T4 levels were observed up to month 10. The measured values for T3 and T4 at 7 and 10 months periods were 2.4±0.3 ng/ml and 160±29 ng/ml, respectively, and these values were consistent at these levels until the beginning of the microemulsion transdermal treatment in month 14 (FIGS. 15A and 15B).

TSH showed an opposite pattern as compared to T3 and T4 during the initial non-treatment period (FIG. 15C). Initially, the TSH plasma values in ID and in control groups were 1.4±0.4 ng/ml and 1.2±0.9 ng/ml, respectively. At the end of first month, the value of TSH increased in ID group to 3.2±1 ng/mL, while it maintained at 1.1±0.9 ng/ml in control group. In the following months, the values of TSH in ID group continued to climb with noticeable retardation at months 7 and 8, while the maximum TSH value was attained at month 9 (7.8±2.6 ng/ml). However; this increase was significant in comparison to control group during the first two months and during the 14 months of the experiments. After month 4, average TSH values remained at 1.5-2 ng/ml in the control group and were maintained during the 17 months study (FIG. 15C).

After the beginning of treatment and during the post treatment month, a sharp increase in T3 and T4 values was observed and continued at similar values during the second month (FIGS. 15A and 15B). T3 levels post treatment increased about 4-fold from 2.5±0.2 ng/ml to 10±2 ng/ml while T4 values increased about 2.25 fold from 160±30 ng/ml to 360±40 ng/ml. At the end of the third month a reduction in both T3 and T4 was observed. The average values for T3 and T4 were 7±1.8 ng/ml and 295±80 ng/ml, respectively. The T3 and T4 post treatment values in ID group were similar to T3 and T4 values measured in the control group. The post treatment TSH values exhibited a reduction in the average values of about 1.6±1 ng/ml during the three month post treatment (FIG. 15C). These values were statistically significant (t-test P<0.0001) as compared to TSH values immediately pretreatment (7.7±1.8 ng/ml). The values of T3 and T4, TSH during the 3 months post treatment were equivalent to the values of control group during the same time frame.

Urine Purification and Sample Pretreatment

Since the majority of iodide absorbed into the body is excreted into the urine, the determination of iodide levels in urine becomes a reliable marker for iodide concentration in the plasma. In this Example, iodide secretion was observed in urine in both ID and control groups using 24 hours urinary collections and expressing only the end of the month measurements.

In brief, stored urine samples at −80° C. were left at room temperature for complete thawing. After thawing, samples were centrifuged at 5000 rpm in accuSpin™ Micro/Micro R Bench top Centrifuge (Fisher Scientific Co.) for 10 minutes to precipitate any suspended particulate materials and 500 μl of clear supernatant were transferred into 2 ml eppendorf vial. One and half milliliter of cold alcohol was added to each sample to precipitate proteins. The samples were centrifuged at 5000 rpm for 10 minutes until clear supernatant was acquired. The supernatant was transferred to glass tubes and placed under ultrapure nitrogen stream in 37° C. water bath until all solvent was evaporated and urinary solid matrix was obtained. The urinary solid material was reconstituted with 500 μL, of 5 mM NaOH and vortexed until all solid materials were dissolved. The reconstituted urine samples were passed through 50 mg/1 ml Hypersep C18 extraction Column (Thermo Scientific) for purification using vacuum. Before use, Hypersep C18 Column were first rinsed with 3 ml methanol and then washed with 6 ml ultrapure water. Extra vacuum was applied to rinse any solvent to ensure that there was no dilution of the sample solution during the pretreatment procedure. Ten samples were purified together using Resprep® 24-Port SPE Manifolds (Restek, Bellefonte, Pa.).

High pressure ion chromatography (Shimadzu, Kyoto, Japan) equipped with a suppressed conductivity detector was used to measure iodide concentration in urine. The instrument consisted of an LC-20AD pump, SIL-20AC HT Auto sampler, CTO-20A column oven, CDD-10A VP conductivity detector, and CBM-20A system controller. The system was connected to SeQuant® CARS™ Suppressor system (Millipore). The suppression system consisted of SeQuant® CARS™ Suppressor and SeQuant® CARS™ Cartridge. SeQuant® SAMS™ Suppressor contains an ion exchange membrane and is used for mobile phase background suppression. The SeQuant® CARS™ Cartridge provides Continuous Auto-Regeneration for the suppressor column. Anion separation was performed on Dionex IonPac AG16/AS16 guard/separation columns (Thermo Scientific, Waltham, Mass.). The injection volume was set to 20 μl. The mobile phase consisted of a mixture of 50% of [1.8 mM sodium carbonate (Na₂CO₃) and 1.7 mM sodium bicarbonate NaHCO₃] and 50% of [5 mM Sodium hydroxide] and was used under isocratic conditions at a flow rate of 1 ml/min. The sodium in the mobile phase was efficiently removed by the suppression method used to reduce noise and improve sensitivity. The mobile phase was prepared using Millipore ultrapure water and was filtered through 0.45 μm cellulose membrane (Millipore) and then degassed under vacuum.

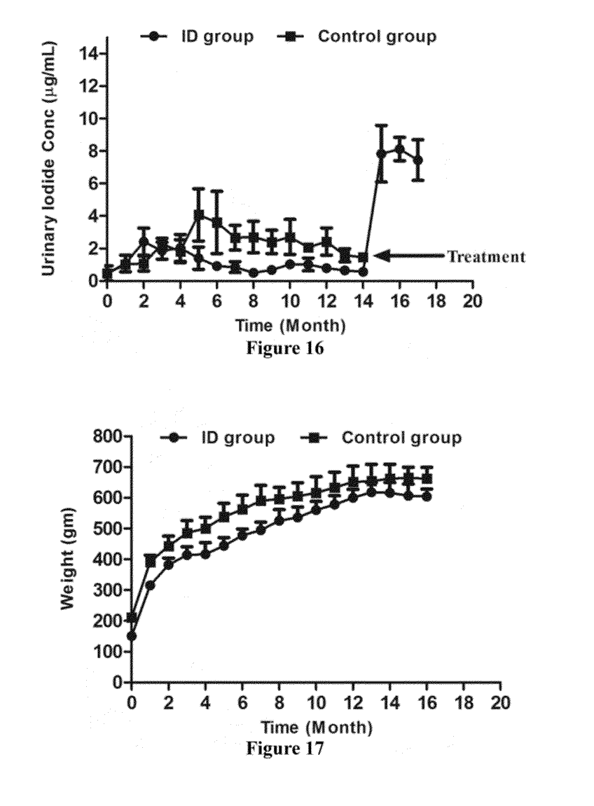

FIG. 16 shows that both ID and control groups had an increasing level of iodide in the first two months of the monitoring period. Iodide concentration increased in both groups from about 0.5±0.5 μg/ml at the beginning of the study to 2±0.8 μg/ml at month 2. There was no statistical significance in iodide concentration between the two groups at this early phase of the study (P>0.05). At the end of month 2, the iodide excretion in urine of ID group declined while the iodide excretion for the control group continued to rise, and statistically significant differences in iodide levels between the two groups were apparent during months 4 to 14 (P<0.0001). The ID group exhibited a slow reduction in iodide level as compared to control at the end of month 2 until it reached a minimum value of 0.5±0.2 μg/ml at the end of month 14, just before the beginning of the treatment phase. In contrast to ID group, iodide levels in control group after month 2 continued to rise reaching average values of 4±2 and 3.6±2 μg/ml during months 5 and 6, respectively. After month 6 iodide concentration started to decrease progressively to reach a value of 1.5±0.1 μg/ml at the end of month 14, but the differences between the two groups remained significant (P<0.05).

Once treatment was applied at month 14 for ID group, there was a sharp increase in iodide levels from 0.5 μg/ml to an average value of 7.8 μg/ml at month 15, and the level remained stable for the following 2 months post treatment. This increase was significantly higher as compared to iodide levels pretreatment, which indicates high levels of iodide in rat plasma.

Body Weight Monitoring of Rats

To determine the influence of iodide deficiency on rat growth rate, the mean body weights (BW) of ID and control groups were monitored throughout the study (FIG. 17). Statistical analysis using Paired t-test was conducted on the weight data for the ID and control groups. The growth rate between the two groups was significantly different (P<0.0001), as the average weight of ID group needed 7 months to reach 500 gm, whereas the rats in control group reached the 500 gm average weight in 3 months (FIG. 17). After 3 months the difference continued and remained significant between the two groups until the end of the monitoring period, thereby indicating slower growth of rats in ID group in compared to control group.

Statistical Analysis

Two-tailed Student's t tests were performed on original data to assess statistical differences between the groups. Significance levels were set at P<0.05.

It will be apparent to those skilled in the art that various modifications and variations can be made in the presently-disclosed subject matter without departing from the scope or spirit of the presently-disclosed subject matter. Other aspects of the presently-disclosed subject matter will be apparent to those skilled in the art from consideration of the specification and practice of the presently-disclosed subject matter. It is intended that the specification and examples be considered as exemplary only, with a true scope and spirit of the presently-disclosed subject matter being indicated by the following claims.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the presently-disclosed subject matter.

As used herein, the term “about,” when referring to a value or to an amount of mass, weight, time, volume, concentration or percentage is meant to encompass variations in some embodiments of ±20%, in some embodiments of ±10%, in some embodiments of ±5%, in some embodiments of ±1%, in some embodiments of ±0.5%, and in some embodiments of ±0.1% from the specified amount, as such variations are appropriate to perform the disclosed method. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Throughout this application, various publications are referenced. All such publications, specifically including the ones listed below, are incorporated herein by reference in their entirety.

### REFERENCES

1. Morgan, D. J. and A. Morgan (1967). “Studies on the Retention and Metabolism of Inhaled Methyl Iodide-I: Retention of Inhaled Methyl Iodide.” Health Physics 13(10): 1055-1065.
2. Risher J F, Keith L S (2009). “Iodine and Inorganic Iodides: Human Health Aspects, 1^{st} ed.” Geneva: WHO Press pp. 14-16.
3. Blum, M. and M. Eisenbud (1967). “Reduction of Thyroid Irradiation From 1311 by Potassium Iodide.” JAMA: The Journal of the American Medical Association 200(12): 1036-1040.
4. Wallace, S. M. and G. Barnett (1978). “Pharmacokinetic analysis of percutaneous absorption; evidence of parallel penetration pathways for methotrexate.” Journal of Pharmacokinetics and Pharmacodynamics 6(4): 315-325.
5. Keister, J. C. and G. B. Kasting (1986). “The use of transient diffusion to investigate transport pathways through skin.” Journal of Controlled Release 4(2): 111-117.
6. Edwards, D. A. and R. Langer (1994). “A linear theory of transdermal transport phenomena.” Journal of Pharmaceutical Sciences 83(9): 1315-1334.
7. Pechtold, L. A. R. M., W. Abraham, et al. (1996). “The Influence of an Electric Field on Ion and Water Accessibility to Stratum Corneum Lipid Lamellae.” Pharmaceutical Research 13(8): 1168-1173.
8. Boddé, H. E., M. A. M. Kruithof, et al. (1989). “Visualisation of normal and enhanced HgCl2 transport through human skin in vitro.” International Journal of Pharmaceutics 53(1): 13-24.
9. Cornwell, P. A. and B. W. Barry (1993). “The routes of penetration of ions and 5-fluorouracil across human skin and the mechanisms of action of terpene skin penetration enhancers.” International Journal of Pharmaceutics 94(1-3): 189-194.
10. Costigan, S. C., P. J. Booth, et al. (2000). “Estimations of lipid bilayer geometry in fluid lamellar phases.” Biochimica et Biophysica Acta (BBA)—Biomembranes 1468(1-2): 41-54.
11. Mitragotri, S. (2003). “Modeling skin permeability to hydrophilic and hydrophobic solutes based on four permeation pathways.” Journal of Controlled Release 86(1): 69-92.
12. Kogan, A. and N. Garti (2006). “Nanoemulsions as transdermal drug delivery vehicles.” Advances in Colloid and Interface Science 123-126: 369-385.
13. Cevc, G. and U. Vierl (2010). “Nanotechnology and the transdermal route: A state of the art review and critical appraisal.” Journal of Controlled Release 141(3): 277-299.
14. Danielsson, I. and B. Lindman (1981). “The definition of nanoemulsion.” Colloids and Surfaces 3(4): 391-392.
15. Lawrence, M. J. and G. D. Rees (2000). “Nanoemulsion-based media as novel drug delivery systems.” Advanced Drug Delivery Reviews 45(1): 89-121.
16. Williams, A. C. and B. W. Barry (2004). “Penetration enhancers.” Advanced Drug Delivery Reviews 56(5): 603-618.
17. McNeill, S. C., R. O. Potts, et al. (1992). “Local enhanced topical delivery (LETD) of drugs: Does it truly exist?” Pharmaceutical Research 9(11): 1422-1427.
18. Dai, G., O. Levy, et al. (1996). “Cloning and characterization of the thyroid iodide transporter.” Nature 379(6564): 458-460.
19. Smanik, P. A., K.-Y. Ryu, et al. (1997). “Expression, Exon-Intron Organization, and Chromosome Mapping of the Human Sodium Iodide Symporter.” Endocrinology 138(8): 3555-3558.
20. Delange F. (1994). “The disorders induced by iodine deficiency.” Thyroid 4: 107-128.
21. Miller L. K, White J. W, Lang M. C, Weidner A. W (1985). “Skin Exposure to I-Blocks Thyroid Uptake of 1311.” Health Physics, 49; 5; 791-794.
22. Zanzonico, P. B. and D. V. Becker (2000). “Effects of Time of Administration and Dietary Iodine Levels on Potassium Iodide (Ki) Blockade of Thyroid Irradiation By 131I From Radioactive Fallout.” Health Physics 78(6): 660-667.
23. Hathout, R. M., T. J. Woodman, et al. (2010). “Nanoemulsion formulations for the transdermal delivery of testosterone.” European Journal of Pharmaceutical Sciences 40(3): 188-196.
24. Monzer, F. (2008). “Phase behavior, transport, diffusion and structural parameters of nonionic surfactants nanoemulsions.” Journal of Molecular Liquids 139(1-3): 14-22
25. Lachenmeier, D. (2008). “Safety evaluation of topical applications of ethanol on the skin and inside the oral cavity.” Journal of Occupational Medicine and Toxicology 3(1): 26.
26. Jaeger, J., K. Sorensen, et al. (1994). “Peroxide accumulation in detergents.” Journal of Biochemical and Biophysical Methods 29(1): 77-81.
27. Lever, M. (1977). “Peroxides in detergents as interfering factors in biochemical analysis.” Analytical Biochemistry 83(1): 274-284.

28. Mancuso, J. R., D. J. McClements, et al. (1999). “Ability of Iron To Promote Surfactant Peroxide Decomposition and Oxidize α-Tocopherol.” Journal of Agricultural and Food Chemistry 47(10): 4146-4149.
29. López, A., F. Llinares, et al. (2000). “Comparative enhancer effects of Span®20 with Tween®20 and Azone® on the in vitro percutaneous penetration of compounds with different lipophilicities.” International Journal of Pharmaceutics 202(1-2): 133-140.
30. Gwak, H. S. and I. K. Chun (2002). “Effect of vehicles and penetration enhancers on the in vitro percutaneous absorption of tenoxicam through hairless mouse skin.” International Journal of Pharmaceutics 236(1-2): 57-64.
31. Jain, J., C. Fernandes, et al. (2010). “Formulation Development of Parenteral Phospholipid-based Nanoemulsion of Etoposide.” AAPS PharmSciTech 11(2): 826-831.
32. Kabalnov, A., U. Olsson, et al. (1995). “Salt Effects on Nonionic Nanoemulsions Are Driven by Adsorption/Depletion at the Surfactant Monolayer.” The Journal of Physical Chemistry 99(16): 6220-6230.
33. De Gennes, P. G. and C. Taupin (1982). “Nanoemulsions and the flexibility of oil/water interfaces.” The Journal of Physical Chemistry 86(13): 2294-2304.
34. Djordjevic, L., M. Primorac, et al. (2004). “Characterization of caprylocaproyl macrogolglycerides based nanoemulsion drug delivery vehicles for an amphiphilic drug.” International Journal of Pharmaceutics 271(1-2): 11-19.
35. Kahlweit, M., R. Strey, et al. (1987). “How to study nanoemulsions.” Journal of Colloid and Interface Science 118(2): 436-453.
36. Yuan, J. S., M. Ansari, et al. (2008). “Linker-based lecithin nanoemulsions for transdermal delivery of lidocaine.” International Journal of Pharmaceutics 349(1-2): 130-143.
37. Handbook of nanoemulsion science and technology, pg370
38. Mathew, C., P. K. Patanjali, et al. (1988). “On the concept of percolative conduction in water-in-oil nanoemulsions.” Colloids and Surfaces 30(3-4): 253-263.
39. Li, X., G. He, et al. (2010). “Study on conductivity property and microstructure of TritonX-100/alkanol/n-heptane/water nanoemulsion.” Colloids and Surfaces A: Physicochemical and Engineering Aspects 360(1-3): 150-158.
40. Jian, X., L. Ganzuo, et al. (2001). “A study of the microstructure of CTAB/1-butanol/octane/water system by PGSE-NMR, conductivity and cryo-TEM.” Colloids and Surfaces A: Physicochemical and Engineering Aspects 191(3): 269-278.
41. Giustini, M., G. Palazzo, et al. (1996). “Microstructure and Dynamics of the Water-in-Oil CTAB/n-Pentanol/n-Hexane/Water Nanoemulsion: A Spectroscopic and Conductivity Study.” The Journal of Physical Chemistry 100(8): 3190-3198.
42. Eicke, H. F., M. Borkovec, et al. (1989). “Conductivity of water-in-oil nanoemulsions: a quantitative charge fluctuation model.” The Journal of Physical Chemistry 93(1): 314-317.
43. Surfactant science and technology. Pg113
44. Yuan, Y., S.-m. Li, et al. (2006). “Investigation of nanoemulsion system for transdermal delivery of meloxicam.” International Journal of Pharmaceutics 321(1-2): 117-123.
45. Lee, P. J., N. Ahmad, et al. (2006). “Evaluation of chemical enhancers in the transdermal delivery of lidocaine.” International Journal of Pharmaceutics 308(1-2): 33-39.
46. Zhang K, Chen M, Scriba G K E, Abraham M H, Fahr A, Liu X. (2012). “Human skin permeation of neutral species and ionic species: Extended linear free-energy relationship analyses.” J Pharm Sci 101: 2034-44.
47. Azeem A, Ahmad F, Khar R, Talegaonkar S. (2009). “Nanocarrier for the Transdermal Delivery of an Antiparkinsonian Drug.” AAPS PharmSciTech 10: 1093-1103.
48. Singh A, Chaurasiya A, Singh M, Upadhyay S, Mukherjee R, Khar R. Exemestane. (2008). “Loaded Self-Microemulsifying Drug Delivery System (SMEDDS): Development and Optimization.” AAPS PharmSciTech 9: 628-634.
49. Zhang Y, Cremer P S. (2006). “Interactions between macromolecules and ions: the Hofmeister series.” Curr Opin Chem Bio. 10: 658-663.
50. Gradzielskin M, Hoffmann H. (1999). “Rheological properties of microemulsions.” In: Kumar P, and Mittal K L (ed.) Handbook of Micromulsion Science and Technology, 1^{st} ed., New York: Marcel Dekker Inc. 1999. p. 357-386.
51. Miot, F., et al., Thyroid hormone synthesis and secretion. Thyroid Managers, 2010: p. 1-38.
52. Zimmermann, M. B., Iodine requirements and the risks and benefits of correcting iodine deficiency in populations. Journal of Trace Elements in Medicine and Biology, 2008. 22(2): p. 81-92.
53. Klein, I. and K. Ojamaa, Thyroid Hormone and the Cardiovascular System. New England Journal of Medicine, 2001. 344(7): p. 501-509.
54. Monaco, F., Thyroid Diseases. 2012: Taylor & Francis.
55. Clero, E., et al., Dietary iodine and thyroid cancer risk in French Polynesia: a case-control study. Thyroid, 2012. 22(4): p. 422-9.
56. Zimmermann, M. B. and M. Andersson, Assessment of iodine nutrition in populations: past, present, and future. Nutrition reviews, 2012. 70(10): p. 553-570.
57. Zimmermann, M. B., Iodine Deficiency. Endocrine Reviews, 2009. 30(4): p. 376-408.
58. Zimmermann, M. B. and M. Andersson, Update on iodine status worldwide. Current Opinion in Endocrinology, Diabetes and Obesity, 2012. 19(5): p. 382-387 10.1097/MED.0b013e328357271a.
59. Abraham, G. E., J. Tlechas, and J. Hakala, Measurement of urinary iodide levels by ion-selective electrode: Improved sensitivity and specificity by chromatography on an ion-exchange resin. The Original Internist, 2004. 11(4): p. 19-32.
60. Serum T S H, T4, and Thyroid Antibodies in the United States Population (1988 to 1994): National Health and Nutrition Examination Survey (NHANES III). The Journal of Clinical Endocrinology & Metabolism, 2002. 87(2): p. 489-499.
61. Berbel, P., et al., Iodine supplementation during pregnancy: a public health challenge. Trends in Endocrinology & Metabolism, 2007. 18(9): p. 338-343.
62. Lou, H., et al., Development of W/O Microemulsion for Transdermal Delivery of Iodide Ions. AAPS PharmSciTech, 2013. 14(1): p. 168-176.
63. Adams, J. F., The Clinical and Metabolic Consequences of Total Gastrectomy. Scandinavian Journal of Gastroenterology, 1968. 3(2): p. 152-159.
64. Schulzke, J.-D., H. Troger, and M. Amasheh, Disorders of intestinal secretion and absorption. Best Practice & Research Clinical Gastroenterology, 2009. 23(3): p. 395-406.
65. Cicalese, M., et al., Requesting iodine supplementation in children on parenteral nutrition. Clinical Nutrition, 2009. 28(3): p. 256-259.
66. Prausnitz, M. R. and R. Langer, Transdermal drug delivery. Nat Biotech, 2008. 26(11): p. 1261-1268.
67. Prausnitz, M. R., S. Mitragotri, and R. Langer, Current status and future potential of transdermal drug delivery. Nat Rev Drug Discov, 2004. 3(2): p. 115-124.
68. Morell, S. F., The Great Iodine Debate. Wise Traditions in Food, Farming, and the Healing Arts, Summer, 2009.
69. Fukuda, H., et al., Changes in plasma thyroxine, triiodothyronine, and TSH during adaptation to iodine deficiency in the rat. Endocrinology, 1975. 97(2): p. 307-314.
70. Versloot, P., et al., Effects of marginal iodine deficiency on thyroid hormone production, distribution and transport in nonpregnant and near-term pregnant rats. European journal of endocrinology, 1998. 138(6): p. 713-718.
71. Kunisue, T., J. W. Fisher, and K. Kannan, Modulation of thyroid hormone concentrations in serum of rats coadministered with perchlorate and iodide-deficient diet. Archives of environmental contamination and toxicology, 2011. 61(1): p. 151-158.
72. Soldin, O. P., Controversies in urinary iodine determinations. Clinical biochemistry, 2002. 35(8): p. 575-579.
73. Jooste, P. L. and E. Strydom, Methods for determination of iodine in urine and salt. Best Practice & Research Clinical Endocrinology & Metabolism, 2010. 24(1): p. 77-88.
74. Zimmermann, M. and F. Delange, Iodine supplementation of pregnant women in Europe: a review and recommendations. European Journal of Clinical Nutrition, 2004. 58(7): p. 979-984.
75. Narayanan, L., et al., Sensitive high-performance liquid chromatography method for the determination of low levels of perchlorate in biological samples. J Chromatogr B Analyt Technol Biomed Life Sci, 2003. 788(2): p. 393-9.
76. Mancini, M., et al., Morphological ultrasound microimaging of thyroid in living mice. Endocrinology, 2009. 150(10): p. 4810-5.
77. VisualSonics, Guide to Small Animal Thyroid Gland Imaging using the Vevo 2100 Imaging System. Imaging Guide: Vevo 2100 Imaging System, 2010.

We claim:

1. A composition, comprising:
an aqueous solution;
a surfactant;
a cosurfactant;
an oil; and
iodine or salts thereof.

2. The composition of claim 1, wherein the iodine or salts thereof is selected from potassium iodide, copper iodide, zinc iodide, sodium iodide, and combinations thereof.

3. The composition of claim 1, wherein the surfactant is selected from anionic surfactants, cationic surfactants, nonionic surfactants, zwitterionic surfactants, and combinations thereof.

4. The composition of claim 1, wherein the surfactant includes sorbitan laurate (Span 20).

5. The composition of claim 1, wherein the cosurfactant includes an alcohol.

6. The composition of claim 5, wherein the cosurfactant includes ethanol.

7. The composition of claim 1, wherein the oil is selected from a volatile oil, a non-volatile oil, and combinations thereof.

8. The composition of claim 1, wherein the oil includes propylene glycol monocaprylate (type II).

9. The composition of claim 1, including sorbitan laurate (Span 20), an alcohol, and propylene glycol monocaprylate (type II).

10. The composition of claim 1, comprising about 0.1 to about 50 wt % aqueous solution, about 0.1 to about 50 wt % surfactant, about 0.1 to about 50 wt % cosurfactant, and about 0.1 to about 50 wt % oil.

11. The composition of claim 1, comprising about 5 to about 25 wt % aqueous solution, about 30 to about 38 wt % surfactant, about 30 to about 38 wt % cosurfactant, and about 15 to about 19 wt % oil.

12. The composition of claim 1, comprising about 0.1 mg/mL to about 500 mg/mL of iodine or salts thereof.

13. The composition of claim 1, wherein the composition is an emulsion.

14. The composition of claim 13, wherein the emulsion includes droplets with an average diameter of about 0.5 nm to about 1000 nm.

15. A method for treating a subject in need thereof, comprising:
administering an effective amount of a composition to the subject, the composition including an aqueous solution, a surfactant, a cosurfactant, an oil, and iodine or salts thereof.

16. The method of claim 15, wherein the administering step includes topically administering the composition to an area of skin on the subject.

17. The method of claim 16, wherein the administering step includes:
providing a spray device that contains the composition; and
actuating the spray device one or more times to topically administer a predetermined volume of the composition to the area of skin on the subject.

18. The method of claim 15, wherein the subject has been diagnosed or prognosed with an iodine deficiency, a thyroid disorder, or a combination thereof.

19. A method of synthesizing a composition, comprising:
mixing a surfactant and a cosurfactant to form a mixture;
adding an oil to the mixture; and
adding an aqueous solution that includes iodine or salts thereof to the mixture.

20. The method of claim 19, wherein the ratio of surfactant to cosurfactant in the mixture is about 10:0 to about 1:9.

21. The method of any of claim 19, wherein the step of adding the aqueous solution is performed after the step of adding the oil.

22. The method of any of claim 19, wherein the step of adding the oil is performed after the step of adding the aqueous solution.

23. The method of any of claim 19, wherein the composition includes about 0.1 to about 50 wt % aqueous solution, about 0.1 to about 50 wt % surfactant, about 0.1 to about 50 wt % cosurfactant, and about 0.1 to about 50 wt % oil

24. The method of any of claim 19, wherein the composition includes about 0.1 mg/mL to about 500 mg/mL iodine or salts thereof.
